(19) United States
(12) Patent Application Publication
(10) Pub. No.: US 2016/0075776 A1
(43) Pub. Date: Mar. 17, 2016

(54) Compositions and Methods for Controlled Release of Agents

(71) Applicants: Paul Ashton, Newton, MA (US); Hong Guo, Wayland, MA (US); Leigh T. Canham, Malvern (GB); Dinesh Nadarassan, Worcestershire (GB)

(72) Inventors: Paul Ashton, Newton, MA (US); Hong Guo, Wayland, MA (US); Leigh T. Canham, Malvern (GB); Dinesh Nadarassan, Worcestershire (GB)

(21) Appl. No.: 14/855,946

(22) Filed: Sep. 16, 2015

Related U.S. Application Data
(60) Provisional application No. 62/050,917, filed on Sep. 16, 2014.

(51) Int. Cl. C07K 16/24; A61K 9/00; C07K 16/22; A61K 38/22; A61K 9/16; C07K 14/715

(52) U.S. Cl. CPC C07K 16/241; A61K 9/16; C07K 14/7151; C07K 16/22; A61K 38/22; A61K 9/0048; C07K 2319/30; C07K 2317/76; C07K 2317/24; C07K 2317/55

(57) ABSTRACT

This invention discloses reservoirs, such as implants for delivering therapeutic agents, particularly large molecules such as proteins and antibodies. The reservoirs may comprise a porous silicon-based carrier material impregnated with the therapeutic agent. The reservoir may be used in vitro or in vivo to maintain an equilibrium concentration of a therapeutic agent over an intended period of time such as over multiple days, weeks, months, or years. Additionally, the reservoir may be reloaded with additional therapeutic agent. These reservoirs may be used for treating or preventing conditions of a subject such as chronic diseases.

### PRIORITY CLAIM

This patent application claims priority to U.S. Provisional Patent Application No. 62/050,917, filed Sep. 16, 2014, which is hereby incorporated by reference in its entirety.

### BACKGROUND

There has been considerable interest within the pharmaceutical industry in the development of dosage forms which provide controlled release of therapeutic agents over a period of time. Releasing an active substance in this way can help to improve bioavailability and ensure that appropriate concentrations of the agent are provided for a sustained period without the need for repeated dosing. In turn, this also helps to minimize the effects of subject non-compliance which is frequently an issue with other forms of administration.

Subjects may be reluctant to comply with their treatment regime, as compliance may be inconvenient, or even painful and traumatic. For example, today there exist therapeutic agents that can treat, with good clinical success, ophthalmic conditions, such as age-related macular degeneration, diabetic macular edema, diabetic retinopathy, choroidal neovascularization, and other conditions that can lead to blindness or near blindness. Often the afflicted population is an older subject group who must adjust their activities of daily living to cope with the early stages of these diseases. However, as the disease progresses, permanent eye damage occurs and many clinically effective treatments are only preventative, and not restorative. Thus, maintaining vision demands consistent compliance with the treatment regime.

Unfortunately, ophthalmic treatment regimens typically require the subject to hold still while the physician pierces the subject's eye with a hypodermic needle to deliver the therapeutic agent into the eye, typically the vitreous of the eye. This can be traumatic and painful and accordingly a subject may be reluctant to receive the injections, which may be required frequently. The ability to provide a longer-term benefit for each injection, and thus reduce the pain and trauma suffered by the subject, turns on the pharmacokinetics of the therapeutic agent and the composition that carries and releases the agent.

Therefore, there remains a continuing need for improved dosage forms for the sustained release of therapeutic agents.

### SUMMARY

Disclosed are porous reservoirs, such as implants, for delivering therapeutic agents, particularly large molecules such as peptides, proteins, antibodies, carbohydrates, polymers, vaccines, small interfering RNA (siRNA), or polynucleotides, in a controlled manner. The reservoirs comprise a porous silicon-based carrier material loaded with the therapeutic agent. In some embodiments, the reservoirs comprise a porous silicon-based carrier material loaded with the therapeutic agent and an amorphous carbohydrate, such as an amorphous sugar. In some embodiments, the reservoirs comprise a porous silicon-based carrier material loaded with the therapeutic agent and a mixture of amorphous sugars. In some embodiments, the reservoirs comprise a porous silicon-based carrier material loaded with the therapeutic agent, and a mixture of a sugar and a crystallization inhibitor. The reservoirs may be used in vitro or in vivo to deliver the therapeutic agent, preferably in a controlled fashion over an intended period of time such as over multiple days, weeks, months, or years. The reservoirs may be formed from a bioerodible or resorbable material, e.g., a silicon-based material such as elemental silicon and/or silicon dioxide, such that removal following release of the therapeutic agent is unnecessary. In certain such embodiments, the reservoir and its breakdown products are biocompatible such that the biological side effects from the bioerosion of the reservoir are minimal or innocuous.

In certain embodiments, the reservoir comprises porous silicon dioxide, such as microporous, mesoporous, or macroporous silicon dioxide or amorphous silica, such as fumed silica. The average pore size of the reservoir is typically selected so that it may carry the therapeutic agent, and example pore sizes are from 2-50 nm in diameter, such as from about 5 to about 40 nm in diameter, from about 15 to about 40 nm in diameter, from about 20 to about 30 nm in diameter, from about 2 to about 15 nm in diameter, or about 5 to about 10 nm in diameter.

In certain embodiments, the therapeutic agent is a protein with a molecular weight between 5,000 amu and 200,000 amu, such as about 10,000 to about 150,000 amu, between 10,000 and 50,000 amu, between 50,000 and 100,000 amu, between 100,000 and 200,000 amu, between 130,000 and 170,000 amu, or between 140,000 and 160,000 amu.

The size of a therapeutic agent may alternatively be characterized by the molecular radius, which may be determined, for example, through X-ray crystallographic analysis or by hydrodynamic radius. The therapeutic agent may be a peptide or protein, e.g., with a molecular radius selected from 0.5 nm to 20 nm, such as about 0.5 nm to 10 nm, even from about 1 to 8 nm. Preferably, a suitable pore radius to allow access to particular agents, e.g., proteins, is selected according to a pore-therapeutic agent (agent) differential, defined herein as the difference between the radius of an agent and a radius of a pore. For example, the pore-agent differential for insulin, with a hydrodynamic radius of 1.3 nm and a pore with a minimum radius of 4.8 nm has a pore-protein differential of 3.5 nm. A pore-agent differential may be used to determine a minimum suitable average pore size for accommodating a protein of a particular radius. The pore-protein differential may typically be selected from about 3.0 to about 5.0 nm.

Typically, the reservoirs are selected to have an average pore size to accommodate the therapeutic agent. The average pore size of the reservoir may be chosen based on the molecular weight or the molecular radius of the therapeutic agent to be loaded into the pores of the reservoir. For example, a therapeutic agent of molecular weight selected from 100,000 to 200,000 amu may be used with a reservoir of larger average pore size such as from about 15 nm to about 40 nm. In certain embodiments, a therapeutic agent of molecular weight selected from 5,000 to 50,000 amu may be used with a reservoir of smaller average pore size such as from about 2 nm to about 10 nm.

The sizes of the pores are selected to maintain a desired equilibrium concentration of a therapeutic agent for a period of time. For example, a pore size may be selected to maintain a concentration of about 1, 2, 3, 4, 5, 6, 7, 8, 9, 10, 11, 12, 13, 14, 15, 16, 17, 18, 19, or 20 μg/ml bevacizumab in the vitreous humor of an eye.

In certain embodiments, the reservoir is loaded with an amorphous carbohydrate, such as an amorphous sugar. In certain embodiments, sugars, used alone or in combination, may be selected from sucrose, fructose, glucose, erythritol, maltitol, lactitol, sorbitol, mannitol, xylitol, D-tagatose, trehalose, trehalose dehydrate, galactose, glycerol, rhamnose, cyclodextrin, raffinose, ribulose, ribose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, xylo-oligosaccharides, gentio-oligoscaccharides, galacto-oligosaccharides, sorbose, nigero-oligosaccharides, fructooligosaccharides, maltotetraol, maltotriol, maltodextrin, malto-oligosaccharides, lactulose, melibiose, or any combinations thereof. In some embodiments, the sugar is selected from trehalose, trehalose dihydrate, sucrose, mannitol, sorbitol, xylitol or glycerol, or a combination thereof. In certain embodiments, the compositions are prepared by forming the porous reservoir first and then loading the pores with the therapeutic agent, the amorphous or solution form of the sugar, or a plurality of sugars, or a combination of a sugar and a crystallization inhibitor. In some embodiments, the therapeutic agent is loaded before the amorphous or solution form of the sugar or the crystallization inhibitor.

In certain embodiments, the reservoirs are prepared by forming the porous reservoir first and then loading the pores with the therapeutic agent.

The invention includes methods for loading a therapeutic agent into the pore of a porous silicon-based reservoir, comprising contacting a porous silicon-based carrier material with a therapeutic agent.

The reservoir may be disposed on the skin or on the surface of the eye. Alternatively, the reservoir may be disposed within the body of a mammal, such as within the eye of a subject, or within any other tissue or organ of the subject's body. In particular applications, the reservoir is disposed subcutaneously, intramuscularly, subconjunctivally, or in the vitreous of the eye. In some applications, the reservoir is disposed in a synovial cavity. The reservoir may be used for treating or preventing conditions of a subject such as chronic diseases. In certain embodiments, the reservoirs are for treating or preventing diseases of the eye such as glaucoma, macular degeneration, diabetic macular edema, age-related macular degeneration, diabetic retinopathy, uveitis, ocular neovascularization, and ocular infection. The reservoirs may also be particularly suitable for use as an ocular reservoir in treating subjects, both human and for veterinarian use, suffering from ocular histoplasmosis, wherein the reservoir may be surgically implanted within the vitreous of the eye. The therapeutic agent may be released in a controlled manner over a period of days, weeks, months, or years, for example, to treat or prevent diseases of the eye such as macular degeneration.

The invention may comprise stabilized formulations and methods of stabilizing therapeutic agents in a porous reservoir as described herein. In certain embodiments, the invention comprises stabilized biomolecules, such as antibodies, in the pores of the reservoir such that the half-life or the shelf life of the biomolecule is superior to the half-life or shelf life of the biomolecule outside of the reservoir. In certain embodiments, the proteins of the stabilized formulations are stable to drying under reduced pressure at room temperature ambient conditions. In certain embodiments, a porous reservoir comprising a therapeutic agent and an amorphous sugar is coated with a polymer, preferably to the extent that the entire surface of the reservoir is coated with the polymer.

The invention may further include a syringe comprising a composition of porous silicon-based carrier material. The syringes may be used to administer a therapeutic agent, such as a peptide or protein, by: a. providing a syringe preloaded with a porous silicon-based reservoir; b. contacting the reservoir with a therapeutic agent; and c. administering the reservoir to the subject. Step b may be carried out by drawing the therapeutic agent into the syringe. Between steps b and c, an incubation time, e.g., 10 min, 20 min, or 30 min, may be taken to allow the therapeutic agent to adsorb into the pores of the reservoir. The therapeutic agent may be a biomolecule, such as a peptide or protein.

In some aspects, the invention relates to methods of reloading a porous drug-delivery reservoir in a subject, comprising administering to a site proximal to the reservoir an agent that has a higher affinity for the reservoir than for surrounding physiological fluid.

In some aspects, the invention relates to methods for manufacturing a porous drug-delivery reservoir, comprising selecting an agent, determining a reservoir pore size that results in a desired equilibrium concentration of the agent in an aqueous solution, and loading a reservoir having pores of the determined pore size with the agent.

In some aspects, the invention relates to methods of delivering a therapeutically effective concentration of an agent to a site in a subject, comprising administering to the site a porous drug-delivery reservoir loaded with the agent, wherein the reservoir has pores configured to maintain a therapeutically effective equilibrium concentration of the agent at the site.

### BRIEF DESCRIPTION OF THE DRAWINGS

FIG. 1 depicts the pore size distribution for a reservoir with a non-uniform, bimodal distribution of pore sizes.

FIG. 2 depicts lysozyme release from various silica matrices in both PBS and SiO₂ saturated PBS. Dissolution medium—PBS: ♦, Davisil 60 Å; ▪, Davisil 150 Å; ▴, Davisil 250 Å. SiO₂ saturated PBS: ⋄, Davisil 60 Å; □, Davisil 150 Å; Δ, Davisil 250 Å.

FIG. 3 depicts cumulative release of bevacizumab from silica adsorbents in phosphate buffered saline.

FIG. 4 depicts the bevacizumab concentration from silicon in which 2 ml of 4 ml media was removed at each time point and replaced with fresh media. The silicon reservoir maintains a bevacizumab concentration of 9.4±2.0 μg/ml.

FIG. 5 depicts the ACTH concentration from silicon in which 2 ml of 4 ml media was removed at each time point and replaced with fresh media. The silicon reservoir maintains an ACTH concentration of 30±4.5 μg/ml.

### DETAILED DESCRIPTION

### Overview

Sustained and controlled delivery of therapeutic agents to subjects, particularly subjects with chronic conditions such as glaucoma or cancer, is becoming increasingly important in modern medical therapy. Many therapies are most effective when administered at frequent intervals to maintain a near constant presence of the active agent within the body, e.g., at an effective concentration that does not elicit undesirable effects incurred at higher concentrations. While frequent administration may be recommended, the inconvenience and associated difficulty of subject compliance may effectively preclude treatment in this manner. As a result, sustained release reservoirs that release therapeutic agents in a controlled manner are very attractive in fields such as cancer therapy and treatment of other chronic diseases. Furthermore, sustained release compositions may allow for dose reduction of the therapeutic agent, thereby leading to reduced side effects.

The sustained release of a therapeutic agent from a reservoir may be achieved by controlling the kinetics of release of the therapeutic agent from a reservoir, for example, by covering the openings of a reservoir with a membrane that slows the rate at which the reservoir releases the agent. Alternatively, sustained release may be achieved by controlling the thermodynamics of release, for example, by providing a reservoir that has an affinity for the therapeutic agent. Thermodynamic control of the release of a therapeutic agent from a reservoir allows the reservoir to maintain an equilibrium concentration of the agent in an aqueous solution. Thus, the reservoir releases the therapeutic agent as the agent is depleted from the surrounding environment, for example, by diffusion or by degradation, to maintain an equilibrium concentration of the agent.

Reservoirs that release therapeutic agents in vivo or in vitro may be formed from a variety of biocompatible or at least substantially biocompatible materials. One type of reservoir employs a silicon-based carrier material. Silicon-based carrier materials may include, for example, elemental silicon, and/or oxidized silicon in forms such as silicon dioxide (silica), or silicates. Some silicon-based carrier materials have demonstrated high biocompatibility and beneficial degradation in biological systems, eliminating the need to remove the reservoir following release of the therapeutic agent.

Tests show that high porosity silicon-based materials, e.g., 80% porosity, are resorbed faster than medium porosity silicon-based material, e.g., 50% porosity, which in turn is resorbed faster than bulk silicon-based material, which shows little to no sign of bioerosion or resorption in biological systems. Furthermore, it is understood that the average pore size of the reservoir will affect the rate of resorption. By adjusting the porosity of the material, the rate of bioerosion may be tuned and selected. The rate of erosion of the silicon can be controlled by controlling the porosity (higher porosity materials are corroded faster) and the barrier thickness.

Silicon-based carrier materials are often prepared using high temperatures and organic solvents or acidic media to form the porous material and load the therapeutic agent within the pores. These conditions may be suitable for certain molecules such as salts, elements, and certain highly stable small organic molecules. However, for loading large organic molecules, such as proteins or antibodies, caustic and/or severe conditions during the preparation or loading of the template could lead to denaturing and deactivation, if not complete degradation of the active agent. Loading large molecules such as antibodies into the reservoir under mild conditions is a feature of the methods described herein that is particularly advantageous for large organic molecules such as proteins.

The particle size of the silicon-based carrier material may also affect the rate at which the pores of the reservoir may be loaded with the therapeutic agent. Smaller particles, e.g., particles in which the largest diameter is 20 microns or less, may load more rapidly than particles in which the largest diameter is greater than 20 microns. This is particularly apparent when the pore diameters are similar in dimensions to the molecular diameters or size of the therapeutic agents. The rapid loading of smaller particles may be attributed to the shorter average pore depth that the therapeutic agent must penetrate in smaller particles and the increased surface area.

### DEFINITIONS

As used herein the specification, “a” or “an” may mean one or more. As used herein in the claim(s), when used in conjunction with the word “comprising”, the words “a” or “an” may mean one or more than one. As used herein “another” may mean at least a second or more.

The terms “antibody” and “antibodies” broadly encompass naturally occurring forms of antibodies and recombinant antibodies, such as single-chain antibodies, camelized antibodies, chimeric, humanized, and human antibodies and multi-specific antibodies as well as fragments and derivatives of all of the foregoing, preferably fragments and derivatives having at least an antigenic binding site. All antibodies are proteins, as that term is used herein. Antibody derivatives may comprise a protein or chemical moiety conjugated to the antibody. The term “antibody” is used in the broadest sense and covers fully assembled antibodies, and recombinant peptides comprising them.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al., Protein Eng. 8(10):1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. In certain embodiments of the invention, the antibody fragment is selected from a Fab, an Fd, an Fd′, a single chain Fv (scFv), an scFvₐ, and a domain antibody (dAb). In other embodiments, the antibody fragment is an Fc portion of an antibody.

The term “aqueous solution” refers to a solution wherein water is a solvent, preferably the primary solvent (e.g., at least 50%, or even more than 80% or more than 90% of the liquid solvents in the solution, such as physiological saline solution). When a porous drug-delivery reservoir of the invention contacts an aqueous solution, it absorbs or releases an agent until an equilibrium concentration of the agent is obtained in the aqueous solution, or until the reservoir is sufficiently saturated with or depleted of the agent or other competing molecules that the reservoir can no longer maintain an equilibrium concentration. The aqueous solution may be a physiological fluid such as the vitreous or aqueous of the eye, the synovial fluid of a synovial joint, or a different body fluid. Alternatively, the aqueous solution may be a buffered solution, such as phosphate-buffered saline. In other embodiments, the aqueous solution may be a solution that comprises the agent, such as a concentrated solution of the agent and one or more stabilizers.

Bioerode or bioerosion, as used herein, refers to the gradual disintegration or breakdown of a structure or enclosure over a period of time in a biological system, e.g., by one or more physical or chemical degradative processes, for example, enzymatic action, hydrolysis, ion exchange, or dissolution by solubilization, emulsion formation, or micelle formation.

The term “carrier material” refers to a porous composition that may be loaded with a given therapeutic agent. A carrier material may comprise one or more particles.

The term “particle” refers to a carrier material i.e. a porous material that may be loaded with a given therapeutic agent.

The term “physiological fluid” refers to bodily fluids, including but not limited to blood, blood plasma, aqueous humor, vitreous humor, and synovial fluid.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of subjects receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

The terms “reservoir” and “implant” are used substantially interchangeably herein to refer to the disclosed materials, with the term “implant” being preferentially used to refer to reservoirs that are implanted into a subject rather than administered by other means. Various descriptions of embodiments of reservoirs are meant to apply equally to implants and vice versa. Reservoirs and implants comprise a carrier material that may be loaded with a given therapeutic agent. The carrier material may comprise one or more particles.

“Resorption” or “resorbing” as used herein refers to the erosion of a material when introduced into or onto a physiological organ, tissue, or fluid of a living human or animal.

Unless otherwise indicated, the term “sugar” refers to monosaccharides, disaccharides, oligosaccharides or sugar alcohols. Examples for the term “sugar” are, but not limited to, sucrose, fructose, glucose, erythritol, maltitol, lactitol, sorbitol, mannitol, xylitol, D-tagatose, trehalose, trehalose dehydrate, galactose, glycerol, rhamnose, cyclodextrin, raffinose, ribulose, ribose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, xylo-oligosaccharides, gentio-oligoscaccharides, galacto-oligosaccharides, sorbose, nigero-oligosaccharides, fructooligosaccharides, maltotetraol, maltotriol, maltodextrin, malto-oligosaccharides, lactulose, melibiose, or any combinations thereof.

The term “surface of a pore wall” refers to a point on the pore wall. An agent interacts with more than one surface of a pore wall if the net interaction energy between the agent and the pore wall is more favorable than the net interaction energy between the agent and a flat surface that has the same chemical properties as the pore wall. The interactions may comprise electrostatic, hydrophobic, and/or van der Waals interactions. For example, a hypothetical agent that can form only two hydrogen bonds might be able to form two hydrogen bonds at the same time with an appropriately-dimensioned pore wall whereas the agent might not be able to form two hydrogen bonds at the same time with a flat surface. Similarly, a flat surface may be able to form two hydrogen bonds with the agent, but an appropriately-dimensioned pore wall may have a more favorable interaction energy, for example, because forming two hydrogen bonds with a flat surface strains the conformation of the agent or limits its conformational entropy relative to an interaction with the pore, or because the pore wall allow for more favorable hydrogen bonding geometries relative to an interaction with a flat surface.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

Unless otherwise indicated, the term large therapeutic molecule refers to molecules with molecular weights equal to or greater than 1000 amu, preferably greater than 2000 amu, or even greater than 3000 amu. Unless otherwise indicated, a small molecule therapeutic molecule refers to a molecule with a molecular weight less than 1000 amu.

As used herein, the term “treating” or “treatment” includes reversing, reducing, or arresting the symptoms, clinical signs, and underlying pathology of a condition in a manner to improve or stabilize a subject's condition.

Silicon-Based Materials and Other Bioerodible Reservoirs

The reservoirs and methods described herein provide, among other things, reservoirs comprising a porous silicon-based carrier material wherein at least one therapeutic agent is disposed in a pore or otherwise adsorbed to a surface of the reservoir. The described methods use such reservoirs for the treatment or prevention of diseases, particularly chronic diseases. Furthermore, the described methods of preparing reservoirs provide reservoirs which are characterized by the capacity to regulate an equilibrium concentration of a therapeutic agent, particularly a large molecule therapeutic agent such as a protein or antibody, in an aqueous environment that surrounds the reservoir.

The reservoir typically comprises a silicon-based carrier material such as elemental silicon, silicon dioxide (silica), silicon monoxide, silicates (compounds containing a silicon-bearing anion, e.g., SiF₆²⁻, Si₂O₇⁶⁻, or SiO₄⁴⁻), or any combination of such materials. The silicon-based carrier material may comprise, for example, semiconducting silicon, elemental silicon, polycrystalline silicon, and/or amorphous silicon. The silicon may be undoped, or may be doped (for example with phosphorus). The silicon-based carrier material may comprise silicon carbide or silicon nitride. In certain embodiments, the silicon-based carrier material comprises a complete or partial framework of elemental silicon and that framework is substantially or fully covered by a silicon dioxide surface layer. In other embodiments, the silicon-based carrier material is entirely or substantially entirely silica. In certain embodiments, the silicon-based carrier material is synthetic amorphous silica. In certain embodiments, the silicon-based carrier material is fumed silica.

Although silicon-based materials are preferred, carrier materials for use in the present invention, additional bioerodible materials with certain common properties (e.g., porosity, pore size, particle size, surface characteristics, bioerodibility, and resorbability) as the silicon-based materials described herein may be used in the present invention. Examples of additional materials that may be used as porous carrier materials are ceramics, metal oxides, semiconductors, bone phosphate, phosphates of calcium (e.g., hydroxyapatite), other inorganic phosphates, carbon black, carbonates, sulfates, aluminates, borates, aluminosilicates, magnesium oxide, calcium oxide, iron oxides, zirconium oxides, titanium oxides, and other comparable materials.

In certain embodiments, the carrier material comprises silica, such as greater than about 50% silica, greater than about 60 wt % silica, greater than about 70 wt % silica, greater than about 80 wt % silica, greater than about 90 wt % silica, greater than about 95 wt % silica, greater than 99 wt % silica, or even greater than 99.9 wt % silica. Porous silica may be purchased from suppliers such as Grace Davison (and sold under the trademark Davisil), Silicycle, and Macherey-Nagel.

In certain embodiments, the carrier material comprises elemental silicon, greater than 60 wt % silicon, greater than 70 wt % silicon, greater than 80 wt % silicon, greater than 90 wt % silicon, or even greater than 95 wt % silicon. Silicon may be purchased from suppliers such as Vesta Ceramics.

Purity of the silicon-based material can be quantitatively assessed using techniques such as Energy Dispersive X-ray Analysis, X-ray fluorescence, Inductively Coupled Optical Emission Spectroscopy, or Glow Discharge Mass Spectroscopy.

The reservoir may comprise other components such as metals, salts, minerals or polymers. The reservoir may have a coating (such as a polymer coating) disposed on at least a portion of the surface, e.g., to improve biocompatibility of the reservoir, to prevent certain molecules from entering the reservoir, or for another reason.

The carrier material may be a porous, amorphous solid or a porous, crystalline solid. For example, the silicon-based carrier material may comprise elemental silicon and/or compounds thereof, e.g., silicon dioxide or silicates, in an amorphous form. In certain embodiments, the elemental silicon or compounds thereof is present in a crystalline form. In other embodiments, the carrier material comprises amorphous silica and/or amorphous silicon. In certain embodiments, the silicon-based material is greater than about 60 wt % amorphous, greater than about 70 wt % amorphous, greater than about 80 wt % amorphous, greater than about 90 wt % amorphous, greater than about 92 wt % amorphous, greater than about 95 wt % amorphous, greater than about 99 wt % amorphous, or even greater than 99.9 wt % amorphous. In certain embodiments, the amorphous silica is fumed silica. In certain embodiments, the amorphous silica is synthetic amorphous silica.

X-ray diffraction analysis can be used to identify crystalline phases of silicon-based material. Powder diffraction can be taken, for example, on a Scintag PAD-X diffractometer, e.g., equipped with a liquid nitrogen cooled germanium solid state detector using Cu K-alpha radiation.

The silicon-based material may have a porosity of about 30% to about 95% such as about 40% to about 95% or 60% to about 80%. Porosity, as used herein, is a measure of the void spaces in a material, and is a fraction of the volume of voids over the total volume of the material. In certain embodiments, the carrier material has a porosity of at least about 10%, at least about 20%, at least about 30%, at least about 40%, at least about 50%, at least about 60%, at least about 70%, at least about 80%, or even at least about 90%. In particular embodiments, the porosity is greater than about 40%, such as greater than about 50%, greater than about 60%, or even greater than about 70%.

The carrier material may have a surface area to weight ratio selected from about 20 m²/g to about 2000 m²/g, such as from about 20 m²/g to about 1000 m²/g, or even from about 100 m²/g to about 300 m²/g. In certain embodiments, the surface area is greater than about 200 m²/g, greater than about 250 m²/g, or greater than about 300 m²/g. In certain embodiments, the surface area is about 200 m²/g.

In certain embodiments, the therapeutic agent is distributed to a pore depth from the surface of the material of at least about 10 microns, at least about 20 microns, at least about 30 microns, at least about 40 microns, at least about 50 microns, at least about 60 microns, at least about 70 microns, at least about 80 microns, at least about 90 microns, at least about 100 microns, at least about 110 microns, at least about 120 microns, at least about 130 microns, at least about 140 microns, or at least about 150 microns. In certain embodiments, the therapeutic agent is distributed in the pores of the reservoir substantially uniformly.

The therapeutic agent may be loaded into the reservoir to a depth which is measured as a ratio of the depth to which the therapeutic agent penetrates the reservoir to the total width of the reservoir. In certain embodiments, the therapeutic agent is distributed to a depth of at least about 10% into the reservoir, to at least about 20% into the reservoir, at least about 30% into the reservoir, at least about 40% into the reservoir, at least about 50% into the reservoir, or at least about 60% into the reservoir.

An amorphous sugar may be loaded into the reservoir to a depth which is measured as a ratio to the total width of the reservoir. In certain embodiments, an amorphous sugar is distributed to a depth of at least about 1%, to at least about 9%, to at least 10% into the reservoir, to at least about 20% into the reservoir, at least about 30% into the reservoir, at least about 40% into the reservoir, at least about 50% into the reservoir, or at least about 60% into the reservoir. In some embodiments, an amorphous sugar may seal the pores.

An amorphous sugar may be loaded into the reservoir to a weight that is measured as a ratio to the combined weight of the reservoir and therapeutic agent. In certain embodiments, an amorphous sugar is loaded to a weight at least about 1% to at least about 80%, at least about 1% to at least about 70%, at least about 1% to at least about 60%, at least about 1% to at least about 50%, at least about 1% to at least about 40%, at least about 1% to at least about 30%, at least about 1% to at least about 20%, to at least about 1% to at least about 15%, about 1% to at least about 10%, about 1% to at least about 5%, about 1% to at least about 4%, at least about 1% to at least about 3%, or at least about 1% to at least about 2%. In certain embodiments, the amorphous sugar is loaded to a weight at least about 5% to at least about 10%, at least about 10% to at least about 20%, at least about 10% to at least about 30%, at least about 30% to at least about 40%, at least about 40% to at least about 50%, at least about 50% to at least about 60%, at least about 60% to at least about 70%, or at least about 70% to at least about 80%. In certain embodiments, an amorphous sugar may be loaded to a weight of about 30%.

Quantification of gross loading may be achieved by a number of analytic methods, for example, gravimetric, EDX (energy-dispersive analysis by x-rays), Fourier transform infra-red (FTIR) or Raman spectroscopy of the pharmaceutical composition or by UV spectrophotometry, titrimetric analysis, HPLC or mass spectroscopy of the eluted therapeutic agent in solution. Quantification of the uniformity of loading may be obtained by compositional techniques that are capable of spatial resolution such as cross-sectional EDX, Auger depth profiling, micro-Raman, and micro-FTIR.

Porous silicon-based materials of the invention may be categorized by the average diameter of the pore size. Microporous silicon-based material has an average pore size less than 2 nm, mesoporous silicon-based material has an average pore size of between 2-50 nm and macroporous silicon-based material has a pore size of greater than 50 nm. In certain embodiments, greater than 50% of the pores of the silicon-based material have a pore size from 2-50 nm, greater than 60% of the pores of the silicon-based material have a pore size from 2-50 nm, greater than 70% of the pores of the silicon-based material have a pore size from 2-50 nm, greater than 80% of the pores of the silicon-based material have a pore size from 2-50 nm, or even greater than 90% of the pores of the silicon-based material have a pore size from 2-50 nm.

In certain embodiments, the carrier material comprises porous silicon dioxide, such as mesoporous silicon dioxide. In certain embodiments, the average pore size of the reservoir is selected from 2-50 nm, such as from about 5 to about 40 nm, from about 15 to about 40 nm, such as about 20 to about 30 nm. In certain embodiments, the average pore size is selected from about 2 to about 15 nm, such as about 5 to about 10 nm. In certain embodiments, the average pore size is about 30 nm.

In certain embodiments, the reservoir has a population of pores with a well-defined pore size, i.e., the distribution of pore sizes for the reservoir falls within a defined range. In certain embodiments, a well-defined population of pores has about 50% to about 99% of the pore sizes within about 1 nm to 15 nm of the average pore size for that population, preferably within about 10 nm, about 5 nm, or even within 3 nm or 2 nm of the average pore size for that population. In certain such embodiments, greater than about 50%, greater than about 60%, greater than about 70%, greater than about 80%, greater than about 90%, or even greater than about 95% of the pores of the reservoir have pore sizes within the specified range. Similarly, a population of pores with a well-defined pore size can be a population in which greater than about 50%, greater than about 60%, greater than about 70%, greater than about 80%, greater than about 90%, or even greater than about 95% of the pores have pore sizes within 20%, preferably within 15%, 10%, or even 5% of the average pore size for that population.

Pore (e.g., mesopore) size distribution can be quantified using established analytical methods such as gas adsorption, high resolution scanning electron microscopy, nuclear magnetic resonance cryoporosimetry and differential scanning calorimetry. In certain embodiments, more than one technique is used on a given sample.

In some embodiments, a population of pores with a well-defined pore size can be a population for which the standard deviation of the pore sizes is less than 20%, preferably less than 15%, less than 10%, or even less than 5% of the average pore size for that population.

The pore size may be preselected to the dimensional characteristics of the therapeutic agent to control the equilibrium concentration of the therapeutic agent in an aqueous solution. Typically, pore sizes that are too small preclude loading of the therapeutic agent, while oversized pores do not interact with the therapeutic agent sufficiently strongly to maintain an equilibrium concentration of the therapeutic agent in an aqueous solution. For example, the average pore diameter for a reservoir may be selected from larger pores, e.g., 15 nm to 40 nm, for high molecular weight molecules, e.g., 200,000-500,000 amu, and smaller pores, e.g., 2 nm to 14 nm, for molecules of a lower molecular weight, e.g., 10,000-50,0000 amu. For instance, average pore sizes of about 6 nm to 12 nm in diameter may be suitable for molecules of molecular weight around 14,000 to 15,000 amu, such as about 14,700 amu. Average pore sizes of about 10 nm to 14 nm in diameter may be selected for molecules of molecular weight around 45,000 to 50,000 amu, such as about 48,000 amu. Average pore sizes of about 15 nm to 30 nm in diameter may be selected for molecules of molecular weight around 150,000 amu.

The pore size may be preselected to be adapted to the molecular radius of the therapeutic agent to control the equilibrium concentration of the therapeutic agent in an aqueous solution. For instance, average pore sizes of about 25 nm to about 40 nm in diameter may be suitable for molecules with a largest molecular radius from about 6 nm to about 8 nm. Molecular radii may be calculated by any suitable method such as by using the physical dimensions of the molecule based on the X-ray crystallography data or using the hydrodynamic radius which represents the solution state size of the molecule. As the solution state calculation is dependent upon the nature of the solution in which the calculation is made, it may be preferable for some measurements to use the physical dimensions of the molecule based on X-ray crystallography data. As used herein the largest molecular radius reflects half of the largest dimension of the therapeutic agent.

In certain embodiments, a reservoir composition comprises two or more different materials with different properties (e.g., pore sizes, particle diameters, or surface characteristics), each preselected to be adapted to a different therapeutic agent. For example, two different reservoirs may be admixed, one with a first population of pores whose pore size is adapted to a first therapeutic agent, the other with a second population of pores whose pore size is adapted to a second therapeutic agent. In some embodiments, the reservoir composition comprises a first population of carrier particles having a first population of pores whose pore size is adapted to a first therapeutic agent, and a second population of carrier particles having a second population of pores whose pore size is adapted to a second therapeutic agent. In certain other embodiments, the reservoir composition comprises a single material that has two or more well-defined populations of pores, e.g., wherein the reservoir is made by a molecular templating technique, such that the characteristics of the pores are preselected for two or more therapeutic agents, e.g., two therapeutic agents with different molecular radii. Thus, the reservoir may deliver two or more therapeutic agents in the controlled manner described herein. In such embodiments, the loading of the therapeutic agents is preferably ordered from the largest to smallest agent, so that the largest agent selectively adsorbs into the largest pores (i.e., it does not fit into the smaller pores), so that the larger pores do not adsorb smaller agents.

For example, if a reservoir composition comprises a first population of well-defined pores that are about 6 nm in diameter (i.e., suitable for molecules of molecular weight around 14,000 to 15,000 amu) and a second population of well-defined pores that are about 10 nm in diameter (i.e., suitable for molecules of molecular weight around 45,000 to 50,000 amu), the latter therapeutic agent (i.e., the one with molecules of molecular weight around 45,000 to 50,000 amu) is preferably added to the reservoir composition prior to adding the smaller therapeutic agent (i.e., the one with molecules of molecular weight around 14,000 to 15,000 amu), although the greater affinity between the smaller agent and the smaller pores may result in equilibration that favors the large agent in the large pores and the small agent in the small pores, regardless of whether the agents are added simultaneously or in any order. Alternatively and additionally, in embodiments wherein the two different porous materials are combined to form a reservoir composition, each reservoir may be separately loaded with a different therapeutic agent and then the reservoirs may be combined to yield the reservoir composition.

In certain embodiments in which the reservoir composition has two or more distinct well-defined populations of pores (e.g., the distinct pore populations are substantially non-overlapping), the differences between the properties of the different populations of pores are preferably selected to limit the adsorption of each different therapeutic agent to a certain population of pores. In certain embodiments, the average pore size of the two or more distinct well-defined pore populations may be selected to limit the adsorption of the larger therapeutic agents into smaller pores. The average pore size differential may be defined as the difference between the average pore sizes for the different populations of pores in the reservoir composition. For example, an average pore size differential of at least 10 nm could indicate that the reservoir composition may comprise at least two populations of pores whose average pore sizes differ (“average pore size differential”) by at least 10 nm., e.g., the composition may comprise two pore populations having average pore sizes of 10 nm and 20 nm, three populations of pores with average pore sizes of 10 nm, 20 nm, and 30 nm, or four populations of pores with average pore sizes of 10 nm, 20 nm, 30 nm, and 40 nm. In certain embodiments, the average pore size differential is preferably at least about 5 nm, at least about 10 nm, at least 15 nm, at least about 20 nm, or at least about 30 nm. In certain embodiments, the two or more well-defined pore populations have distinct average pore sizes, such that the average pore sizes of any two populations differ by at least 20%, preferably at least 30%, 40%, or even 50% of the smaller average pore size.

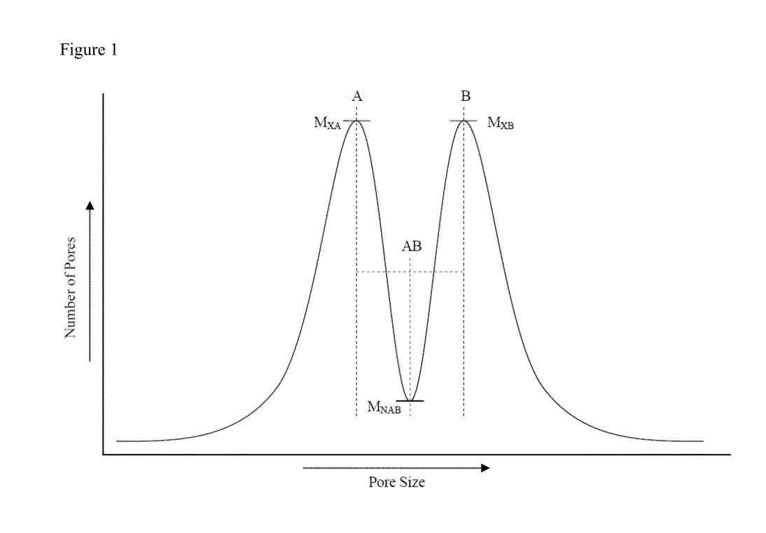

In certain embodiments in which the reservoir composition has a non-uniform distribution of pore sizes, the reservoir composition has two or more well-defined populations of pores with distinct average pore sizes as described above. Similarly, by reference to FIG. 1, a reservoir composition with a non-uniform distribution of pore sizes can be characterized as having a distribution of pore sizes having at least two local maxima (e.g., one at pore size equal to A and one at pore size equal to B in FIG. 1), but as many as three or four local maxima, wherein the number of pores having the size of two adjacent local maxima (e.g., M_{XA} and M_{XB} in FIG. 1) is at least three times, but preferably five times, ten times, or even 20 times the number of pores having a pore size that is the average of the pore sizes of the two local maxima (e.g., M_{NAB} in FIG. 1, wherein the average of the pore sizes of the two local maxima is AV_{AB}). The distribution of pore sizes may also be described by the following equations, which also apply in certain embodiments wherein M_{XA} are M_{XB} are not equivalent, e.g., the distribution is not strictly bimodal:

M_{XA}≧3(M_{NAB}) and M_{XB}≧3(M_{NAB}),

wherein M_{XA}=# of particles of pore size A; M_{XB}=# of particles of pore size B; and M_{NAB}=# of particles of pore size (A+B)/2, and where the 3 may be replaced by any suitable multiplier as described above.

In some embodiments, the reservoir can store and deliver a therapeutically effective amount of an agent. In preferred embodiments, the agent is a therapeutic. As used herein, the term “therapeutic” encompasses the active molecule as well as salts of the active molecule. The therapeutic agent may be, for example, a drug or a prodrug.

In certain embodiments, the therapeutic agent is selected from any agent useful in the treatment or prevention of diseases. In certain embodiments, the agent is selected from small molecule therapeutic agents, i.e., compounds with molecular weights less than 1000 amu. In preferred embodiments, the therapeutic agents are selected from large molecules with molecular weight equal to or greater than 1000 amu. In certain embodiments, the therapeutic agent of the invention is a biomolecule. Biomolecules, as used herein, refer to any molecule that is produced by a living organism, including large polymeric molecules such as proteins, polysaccharides, and nucleic acids as well as small molecules such as primary metabolites, secondary metabolites, and natural products or synthetic variations thereof. In particular, proteins such as antibodies, ligands, and enzymes may be used as therapeutic agents of the invention. In some embodiments, the agent is monomeric insulin, and the pore size is about 5 nm to about 8 nm. In particular embodiments, the biomolecules of the invention have molecular weights ranging from about 10,000 amu to about 500,000 amu. In certain embodiments, the therapeutic agent is a protein such as an antibody. In some embodiments, the antibody is a monoclonal antibody. The agent may comprise an antigen-binding portion of a full-length antibody, e.g., a Fab fragment or a single chain variable fragment, or an Fc fragment of an antibody. Particular agents that may be delivered by the reservoir include Fab fragment ranibizumab (Lucentis), the therapeutic antibody bevacizumab (Avastin), and the fusion protein aflibercept (Eylea).

Suitable peptides and proteins for use in the reservoirs described herein (e.g., reservoirs comprising porous silicon-based carrier particles) include insulin, growth hormones, insulin related growth factor, heat shock proteins, and analogs, derivatives, pharmaceutically acceptable salts, esters, prodrugs, and protected forms thereof.

Polynucleotides that may be administered using the reservoirs herein include DNA, RNA, and analogs of DNA and RNA. For example, the polynucleotides may include 2′O-Me nucleotides or dideoxynucleotides. The polynucleotides may encode proteins for gene therapy, or may be designed to reduce expression of a target gene via an antisense pathway.

In certain embodiments, the therapeutic agent has a molecular weight between 10,000 and 50,000 amu, between 50,000 and 100,000 amu or between 100,000 and 150,000 amu. In certain embodiments, the therapeutic agent is a protein with a molecular weight between 5,000 amu and 200,000 amu, such as about 10,000 to about 150,000 amu.

The size of a therapeutic agent may alternatively be characterized by the molecular radius, which may be determined, for example, through X-ray crystallographic analysis or by hydrodynamic radius. The therapeutic agent may be a peptide or protein, e.g., with a molecular radius selected from 0.5 nm to 20 nm such as about 0.5 nm to 10 nm, even from about 1 to 8 nm.

A therapeutic agent with molecular radius from 1 to 2.5 nm may be advantageously used with a reservoir with a minimum pore radius of from 4.5 to 5.8 nm. A therapeutic agent with a molecular radius of 7 nm may be advantageously used with a reservoir with a minimum pore radius of from 11 to 13 nm, such as about 12 nm. For example, insulin with a hydrodynamic radius of 1.3 nm may be used with a reservoir that has an average minimum pore radius of 4.8 nm.

The protein-pore differential may be used to aid the selection of a suitable reservoir to accommodate the therapeutic agent. This calculation subtracts the molecular radius from the pore radius. Typically, the radius of the therapeutic agent would be the hydrodynamic radius or largest radius determined through x-ray crystallographic analysis. The pore radius would typically be the average pore radius of the reservoir. For example, the pore-protein differential for insulin, with a hydrodynamic radius of 1.3 nm and a pore with a minimum radius of 4.8 nm has a protein-pore differential of 3.5 nm. In certain embodiments, the protein-pore differential is selected from 3 to 6 nm, such as from 3.2 to 4.5 nm. The protein-pore differential may be about 3.2 nm, about 3.3 nm, about 3.4 nm, about 3.5 nm, about 3.6 nm, about 3.7 nm, about 3.8 nm, about 3.9 nm, about 4.0 nm, about 4.1 nm, about 4.2 nm, about 4.3 nm, about 4.4 nm or about 4.5 nm.

In certain embodiments, the therapeutic agent is an antibody and the average pore size of the reservoir is selected from about 5 nm to about 40 nm, for instance about 10 nm to about 40 nm, such as about 10 nm to about 30 nm, such as from about 15 nm to 30 nm. In certain embodiments, the therapeutic agent is an antibody selected from bevacizumab, aflibercept, or ranibizumab and the average pore size of the reservoir is selected from about 5 nm to about 40 nm, such as 7 nm to about 40 nm, such as from about 7 nm to 25 nm. In certain embodiments, the therapeutic agent is bevacizumab and the average pore size of the reservoir is about 15 nm to 25 nm. In certain embodiments, the therapeutic agent is aflibercept and the average pore size of the reservoir is about 15-25 nm, for example, 17 nm to 19 nm. In certain embodiments, the therapeutic agent is ranibizumab and the average pore size of the reservoir is about 7 nm to 16 nm, for example, 7 nm to 14 nm.

In certain embodiments, the walls of the reservoir that separate the pores have an average width of less than 5 nm, such as about 4.8 nm, about 4.6 nm, about 4.4 nm, about 4.2 nm, about 4.0 nm, about 3.8 nm, about 3.6 nm, about 3.4 nm, about 3.2 nm, about 3.0 nm, about 2.8 nm, or even about 2.6 nm. In certain embodiments, the walls of the reservoir that separate the pores have an average width of less than about 3 nm, such as about 2.8 nm, about 2.6 nm, about 2.4 nm, about 2.2 nm, about 2.0 nm, about 1.8 nm, about 1.6 nm, about 1.4 nm, about 1.2 nm, about 1.0 nm, or even about 0.8 nm.

Dimensionality and morphology of the reservoir can be measured, for example, by Transmission Electron Microscopy (TEM) using a 2000 JEOL electron microscope operating, for example, at 200 keV. Samples for TEM can be prepared by dispensing a large number of porous reservoirs onto a holey carbon film on a metal grid, via a dilute slurry.

In certain embodiments, the pores of the reservoir define a space having a volume of about 0.1 mL/g to about 5 mL/g of the reservoir. In certain embodiments, the pore volume is about 0.2 mL/g to about 3 mL/g, such as about 0.4 mL/g to about 2.5 mL/g, such as about 1.0 mL/g to about 2.5 mL/g.

In certain embodiments, the load level of the reservoir is up to 70%, such as up to 40% by weight based on the combined weight of the reservoir and the therapeutic agent. The load level is calculated by dividing the weight of the loaded therapeutic agent by the combined weight of the loaded therapeutic agent and reservoir and multiplying by 100. In certain embodiments, the load level of the reservoir is greater than 1%, such as greater than 2%, greater than 3%, greater than 5%, greater than 10%, greater than 15%, greater than 20%, greater than 25%, greater than 30%, greater than 35%, greater than 40%, greater than 45% or greater than 50%. In certain embodiments, the load level of the reservoir is less than 5%, or between about 4% and about 6%. The load level may be between about 5% and about 10%. In certain embodiments, the load level of the reservoir is between about 10% and about 20%, between about 20% and about 30%, between about 30% and about 40%, between about 40% and about 50%, or between about 50% and about 60%, between about 60% and about 70%, or between about 70% and about 80% by weight.

The composition may comprise the reservoir, the therapeutic agent, the amorphous sugar and optionally other components such as a crystallization inhibitor. In some embodiments, the composition comprises: a therapeutic agent (such as a protein) in the range of 1% to 40% by weight; an amorphous sugar in the range of 1% to 50% by weight; and a reservoir in the range of 10% to 30% by weight.

The load volume of the reservoirs described herein may be evaluated in terms of the volume of the pores in the porous material being occupied by the therapeutic agent. The percentage of the maximum loading capacity that is occupied by the therapeutic agent (that is, the percentage of the total volume of the pores in the porous reservoir that is occupied by the therapeutic agent) for reservoirs according to the invention may be from about 30% to about 100%, such as from about 50% to about 90%. For any given reservoir, this value may be determined by dividing the volume of the therapeutic agent taken up during loading by the void volume of the reservoir prior to loading and multiplying by one hundred.

In certain embodiments, the reservoirs of the invention are particles that, measured at the largest diameter, have an average size of about 1 to about 500 microns, such as about 5 to about 100 microns. In certain embodiments, a single reservoir measured at its largest diameter is about 1 to about 500 microns, such as about 5 to about 500 microns, or about 2 to about 100 microns. In certain embodiments, at least 80%, 90%, 99%, or even 100% of the particles in the reservoir, measured at the largest diameter, are about 1 to about 500 microns, such as about 5 to about 500 microns, or about 2 to about 100 microns.

In order to increase the rate of loading of the particles of the invention, it may be advantageous to use relatively small particles. As smaller particles have pores with less depth for the therapeutic agent to penetrate, the amount of time needed to load the particles may be reduced. This may be particularly advantageous when the pore diameters are similar in dimensions to the molecular diameters or size of the therapeutic agents. Smaller particles may be from 1-20 microns, such as about 10-20 microns, e.g., about 15-20 microns, measured at the largest dimension.

In some aspects, greater than 60%, greater than 70%, greater than 80% or greater than 90% of the particles have a particle size of from 1-20 microns, preferably 5-15 microns, measured at the largest dimension. The particles may have an average particle size between 1 and 20 microns such as between 5-15 microns or about 15 microns, about 16 microns, about 17 microns, about 18 microns, about 19 microns.

Particle size distribution, including the mean particle diameter can be measured, for example, using a Malvern Particle Size Analyzer, Model Mastersizer, from Malvern Instruments, UK. A helium-neon gas laser beam may be projected through an optical cell containing a suspension of the reservoir. Light rays striking the reservoir are scattered through angles which are inversely proportional to the particle size. The photodetector array measures the light intensity at several predetermined angles and electrical signals proportional to the measured light flux values are then processed by a microcomputer system against a scatter pattern predicted from the refractive indices of the sample reservoir and aqueous dispersant.

Larger reservoirs are also envisioned for the delivery of therapeutic agents. The reservoirs of the invention may have an average size of about 1 mm to about 5 cm measured at the largest dimension. In certain embodiments, the reservoirs have an average size of about 5 mm to about 3 cm measured at the largest dimension. Reservoirs greater than 1 mm, as measured at the largest dimension, may be useful for intramuscular, subcutaneous, intravitreal, synovial, or subdermal drug delivery.

In certain embodiments, the agent is prone to decomposition and/or inactivation, and the reservoir reduces decomposition/inactivation of the agent. The agent can be inactivated, for example, by degradation or unfolding/denaturation. For instance, the agent might be prone to inactivation during the process of curing of a polymer (e.g., with heat or ultraviolet light) and the reservoir reduces this inactivation. For instance, the reservoir may block the condition that inactivates the agent (e.g., by absorbing the wavelength of light used for curing). As another example, the carrier may stabilize an agent against the effects of a condition that inactivates the agent, e.g., by stabilizing the agent in an active conformation and restricting protein unfolding or degradation. In some embodiments, the agent experiences at least twice, five times, ten times, 20 times, 50, times or 100 times as much inactivation (e.g., degradation or unfolding) without the reservoir as the agent within the reservoir under the same conditions during production of the reservoir.

In certain embodiments, the porous reservoirs described herein are used to stabilize sensitive therapeutic compounds, such as biomolecules, e.g., antibodies. In certain embodiments, the amorphous sugars described herein present in the pores are used to stabilize sensitive therapeutic compounds, such as biomolecules, e.g., antibodies. In certain embodiments, biomolecules that are partially or wholly unstable at elevated temperatures, such as room temperature or above, can be made stable at room temperature for prolonged periods of time. For example, the biomolecule formulated with one or more amorphous sugars within the reservoir is stable to drying under reduced pressure at room temperature. Similarly, the biomolecule formulated with one or more amorphous sugars within the reservoir may display increased stability during the manufacturing and/or storage of the device. For example, an amorphous sugar may stabilize the biomolecule in the presence of an organic solvent, such as dimethyl sulfoxide (DMSO). In certain embodiments, biomolecules that are partially or wholly unstable at elevated temperatures, such as room temperature or above, can be made stable at room temperature for prolonged periods of time. The biomolecules may be loaded into a reservoir such that an aqueous suspension of the biomolecule loaded into the reservoir is more stable than a corresponding aqueous solution of the biomolecule (i.e., an identical aqueous solution with and without the addition of the porous reservoir). For example, the biomolecule within the reservoir may have a half-life at room temperature (e.g., about 23° C.) that is greater than a half-life of the biomolecule without the reservoir under the same conditions. In certain embodiments, a biomolecule in the pores of the reservoir has a half-life that is at least twice as long as the biomolecule outside of the reservoir under the same conditions, more preferably, at least 5 times, at least 10 times, at least 15 times, at least 20 times, at least 30 times, at least 40 times, at least 50 times, at least 60 times, or at least 100 times as long as the biomolecule outside of the reservoir. For example, an antibody within the pores of the reservoir may have a half-life that is at least 10 times as long as the antibody outside of the reservoir, more preferably, at least 20 times as long.

Similarly, biomolecules may have a longer shelf life within the pores of the reservoir than in a corresponding aqueous solution, preferably at least twice as long, at least five times as long, at least 10 times as long, at least 20 times as long, at least 30 times as long, at least 40 times as long, at least 50 times as long, at least 60 times as long, or at least 100 times as long. For example, an antibody within the pores of the reservoir may have a longer shelf life than an antibody outside of the reservoir, preferably at least 10 times as long or at least 20 times as long.

In certain embodiments, porous reservoirs comprising a carrier material and a biomolecule, such as an antibody, exhibit stability at the temperature of 25° C. for at least 15 days, or even about 1 month. Additionally or alternatively, in certain embodiments, the antibody-loaded reservoirs are stable at 25° C. for at least 6 months, at least 1 year, at least 1.5 years, at least 2 years, at least 2.5 years, at least 3 years, or at least 4 years. Stability may be assessed, for example, by high performance size exclusion chromatography (HPSEC) or by comparing the biological activity of the stored biomolecule-loaded reservoirs against a sample of freshly prepared biomolecule-loaded reservoirs or against the activity of the biomolecules as measured prior to storage. Activity of antibodies, for example, can be assessed by various immunological assays including, for example, enzyme-linked immunosorbent assay (ELISA) and radioimmunoassay. Preferably, at the end of the storage period, the activity of the stored reservoirs is at least 75%, at least 80%, at least 85%, at least 90%, at least 95%, at least 98%, at least 99%, at least 99.5%, at least 99.8%, or even at least 99.9% of the activity of the corresponding freshly prepared reservoirs. Accordingly, this disclosure contemplates methods of treatment wherein biomolecule-loaded reservoirs are stored at 25° C. for at least 6 months, at least 1 year, at least 1.5 years, at least 2 years, at least 2.5 years, at least 3 years or at least 4 years prior to administering the reservoirs to a subject. In some embodiments, the degradation-sensitive agent is a biomolecule, such as a protein, including an antibody.

The invention further comprises methods of stabilizing biomolecules. Methods of the invention comprise loading biomolecules into the pores of the carrier material through any suitable method to form the reservoirs of the invention.

In certain embodiments, the reservoir also comprises one or more pharmaceutically acceptable excipients. In some embodiments, the excipient is a filler, binder, diluent, buffering agent, moistening agent, preservative, stabilizer, flavoring agent, dye, coloring agent, disintegrating agent, or surfactant. In some embodiments, a buffering agent is used to tailor the affinity of the therapeutic agent for the reservoir by creating a micro-environment pH in the reservoir. Thus, the pH can affect the equilibrium concentration of the therapeutic agent in an aqueous solution. A surfactant may be used to adjust the charge, lipophilicity or hydrophilicity of the carrier, such as to enhance wettability of poorly soluble or hydrophobic compositions. Some examples of materials which can serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) hydrophobic materials such as cocoa butter, suppository waxes, and the like; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; and (16) other non-toxic compatible substances employed in pharmaceutical formulations. The excipient may be disposed within pores of the reservoir. In other embodiments, the excipient is outside of the particles. For instance, the particles may be suspended in solution and/or form a slurry, and the excipient may be in the solution.

Methods of Preparation

The invention also provides methods of preparing silicon-based carrier materials. In certain embodiments, porous silicon-based carrier material may be prepared synthetically. For example, porous silica may be synthesized by reacting tetraethyl orthosilicate with a template made of micellar rods. In certain embodiments, the result is a collection of spheres or rods that are filled with a regular arrangement of pores. The template can then be removed, for example, by washing with a solvent adjusted to the proper pH. In certain embodiments, the porous silicon-based carrier material may be prepared using a sol-gel method or a spray drying method. In certain embodiments, the porous silicon based carrier material may be prepared by flame hydrolysis of silicon tetrachloride in an oxy-hydrogen flame. In certain embodiments, the preparation of the carrier material involves one or more techniques suitable for preparing porous silicon-based material.

Pores may be introduced to the silicon-based carrier material through techniques such as anodization, stain etching, or electrochemical etching. In exemplary embodiments, anodization employs a platinum cathode and silicon wafer anode immersed in hydrogen fluoride (HF) electrolyte. Corrosion of the anode producing pores in the material is produced by running electrical current through the cell. In particular embodiments, the running of constant direct current (DC) is usually implemented to ensure steady tip-concentration of HF resulting in a more homogeneous porosity layer.

In certain embodiments, pores are introduced to the silicon-based carrier material through stain-etching with hydrofluoric acid, nitric acid and water. In certain embodiments, a combination of one or more stain-etching reagents is used, such as hydrofluoric acid and nitric acid. In certain embodiments, a solution of hydrofluoric acid and nitric acid are used to form pores in the silicon-based material.

The porosity of the material can be determined by weight measurement. BET analysis may be used to determine any one or more of the pore volume, pore size, pore size distribution and surface area of the carrier material. BET theory applies to the physical adsorption of gas molecules on a solid surface and serves as the basis for an important analysis technique for the measurement of the specific surface area of a material. The BET analysis may be performed, for example, with a Micromeritics ASAP 2000 instrument available from Micromeritics Instrument Corporation, Norcross, Ga. In an exemplary procedure, the sample of the carrier material may be outgassed under vacuum at temperatures, for example, greater than 200° C. for a period of time such as about 2 hours or more before the measurements are taken. In certain embodiments, the pore size distribution curve is derived from the analysis of the adsorption branch of the isotherm output. The pore volume may be collected at the P/P₀=0.985 single point.

One or more drying techniques may be used in the preparation of porous silicon-based materials of the invention. For example, to prevent cracking of the porous silicon-based material, the material may be dried by supercritical drying, freeze drying, pentane drying or slow evaporation, spray drying or vacuum-assisted flash drying. Supercritical drying involves superheating the liquid pore above the critical point to avoid interfacial tension. Freeze drying involves freezing and subliming any solvents under vacuum. Pentane drying uses pentane as the drying liquid instead of water and as a result may reduce capillary stress due to the lower surface tension. Slow evaporating is a technique which can be implemented following the water or ethanol rinsing and may be effective at decreasing the trap density of solvent within the material. Spray drying is a technique whereby a solution of protein and sugar is spray dried so that the water is evaporated sufficiently quickly to allow the sugar to go from a solution to a solid without reordering into a crystal. Vacuum-assisted flash drying is a technique whereby the porous matrix assists the rapid drying of the formulation under reduced pressure whilst stabilizing the amorphous sugar. Vacuum-assisted flash drying may be performed at room temperature, which is desirable for physically stabilized amorphous systems such as biomolecules and sugars.

The surface of the porous silicon-based material may be modified to exhibit properties such as improved stability, cell adhesion or biocompatibility. Optionally, the material may be exposed to oxidizing conditions such as through thermal oxidation. In exemplary embodiments, the process of thermal oxidation involves heating the silicon-based material to a temperature above 1000° C. to promote full oxidation of the silicon-based material. Alternatively, the surface of the carrier material may be oxidized so that the reservoir comprises a framework of elemental silicon partially, substantially or fully covered by an oxidized surface such as a silicon dioxide surface.

The surface of the porous silicon-based material or a portion thereof may be derivatized. In exemplary embodiments, the surface of a porous silicon-based material may be derivatized with organic groups such as alkanes or alkenes. In particular embodiments, the surface of the carrier material may be derivatized by hydrosilation of silicon. In particular embodiments, the derivatized carrier material may function as biomaterials, incorporating into living tissue. Any one or more of electrostatic interactions, capillary action and hydrophobic interactions may enable loading of the therapeutic agent into the pores of the carrier material. In certain embodiments, the carrier material and therapeutic molecules are placed in a solution and the large molecules, e.g., proteins or other antibodies, are drawn from the solution into the pores of the carrier material, reminiscent of a molecular sieve's ability to draw water from an organic liquid. Hydrophobic drugs may be better suited for loading into carrier materials that are predominantly formed from silicon (e.g., greater than 50% of the material is silicon) while hydrophilic drugs may be better suited for loading into a carrier material that is characterized as mostly silica (e.g., greater than 50% of the carrier material is silica). In certain embodiments, the loading of large molecules into the pores of the carrier material is driven by external factors such as sonication or heat. The carrier material, or portion thereof, may have an electrostatic charge and/or the therapeutic agent, or portion thereof, may have an electrostatic charge. Preferably, the carrier material, or portion thereof, has the opposite electrostatic charge as the therapeutic agent, or portion thereof, such that adsorption of the therapeutic agent into the pores of the carrier material is facilitated by the attractive electrostatic forces. In certain embodiments, the therapeutic agent or the carrier material may not have an electrostatic charge by itself, but is instead polarizable and has its polarity modified in the proximity of the carrier material or the therapeutic agent, respectively, which facilitates the adsorption of the therapeutic agent in the pores of the carrier material.

For example, in the body, at physiological pH, silicon dioxide, such as mesoporous silicon dioxide or amorphous silica, exhibits a negatively charged surface, which promotes electrostatic adsorption of positively charged peptides. ACTH and its synthetic analogs, such as cosyntropin, engage in this kind of electrostatic interactions because of the positively charged Lys(15)-Lys(16)-Arg(17)-Arg(18) sequence in their structures. Similarly, molecules with carboxylic acids, phosphoric, and/or sulfonic acids are ionized with increasing pH to negatively charged carboxylate, phosphate, and/or sulfonate salts, while nitrogenated molecules (e.g., bearing amine, guanidine, or other basic substituents) are protonated with decreasing pH to ammonium, guanidinium, or other positively charged salts.

The reservoir may comprise a coating or surface modification to attract the therapeutic agent into the pores. In certain embodiments, the reservoir is coated or modified in whole or in part with a material comprising moieties that are charged in order to attract a protein or antibody into the pores of the reservoir. In other embodiments, the moieties may be appended directly to the reservoir. For example, amine groups may be covalently appended onto the surface of the reservoir such that when protonated at physiological pH, the surface of the reservoir carries a positive charge, thereby, for example, attracting a protein or antibody with a negatively charged surface. In other embodiments, the reservoir may be modified with carboxylic acid moieties such that when deprotonated at physiological pH, the reservoir carries a negative charge, thereby attracting proteins or antibodies with positively charged surfaces into the pores. Such coatings or surface modifications may thereby affect the affinity of the therapeutic agent for the reservoir and alter the equilibrium concentration that the reservoir maintains in an aqueous solution.

In certain embodiments, the carrier material may be a material other than porous silica. Although silicon-based materials are preferred carrier materials for use in the present invention, additional bioerodible materials with certain properties (e.g. porosity, pore size, particle size, surface characteristics, bioerodibility, and resorbability) in common with the silicon-based materials described herein may be used in the present invention. Examples of additional materials that may be used as carrier materials are bioerodible ceramics, bioerodible metal oxides, bioerodible semiconductors, bone phosphate, phosphates of calcium (e.g. hydroxyapatite), other inorganic phosphates, porous carbon black, carbonates, sulfates, aluminates, borates, aluminosilicates, magnesium oxide, calcium oxide, iron oxides, zirconium oxides, titanium oxides, and other comparable materials. Many of these porous materials can be prepared using techniques (e.g., templating, oxidation, drying, and surface modification) that are analogous to the aforementioned techniques used to prepare porous silicon-based carrier materials.

In certain embodiments, the therapeutic agent may be incorporated into the carrier material following complete formation of the reservoir. Alternatively, the therapeutic agent may be incorporated into the carrier material at one or more stages of preparation of the reservoir. For example, the therapeutic agent may be introduced to the carrier material prior to a drying stage of the reservoir, or after the drying of the reservoir or at both stages. In certain embodiments, the therapeutic agent may be introduced to the carrier material following a thermal oxidation step of the carrier material. In certain aspects, the therapeutic agent is introduced as the final step in the preparation of the reservoirs.

More than one therapeutic agent may be incorporated into a carrier material. In certain such embodiments, each therapeutic agent may be individually selected from small organic molecules and large molecules such as proteins and antibodies. For example, an ocular reservoir may be impregnated with two therapeutic agents for the treatment of glaucoma, or one therapeutic agent for the treatment of macular degeneration and another agent for the treatment of glaucoma. In other embodiments, more than one therapeutic agent may be incorporated into a plurality of compositions. For example, two ocular delivery vehicle compositions may be impregnated with a therapeutic agent for the treatment of glaucoma, wherein one delivery vehicle composition is administered at the back of the eye and the other is administered at the front of the eye.

In certain aspects, e.g., when both small molecule therapeutic agents and larger molecular therapeutic agents such as proteins are incorporated into a carrier material, the therapeutic agents may be incorporated into the carrier material at different stages of the preparation of the reservoir. For example, a small molecule therapy may be introduced into the carrier material prior to an oxidation or drying step and a large molecule therapeutic agent may be incorporated following an oxidation or drying step. Similarly, multiple different therapeutic agents of the same or different types may be introduced into a finished reservoir in different orders or essentially simultaneously. When a reservoir comprises a single material, or combination of multiple materials, with multiple pore sizes the larger therapeutic agent is preferably added to the reservoir prior to adding the smaller therapeutic agent to avoid filling the larger pores with the smaller therapeutic agent and interfering with adsorption of the larger therapeutic agent, although the greater affinity between the smaller agent and the smaller pores may result in equilibration that favors the large agent in the large pores and the small agent in the small pores, regardless of whether the agents are added simultaneously or in any order. For example, if a reservoir comprises a single material, or combination of multiple materials, that has some well-defined pores that are about 8 nm to 12 nm in diameter (i.e., suitable for molecules of molecular weight around 14,000 to 15,000 amu) and some well-defined pores that are about 10 nm to 15 nm in diameter (i.e., suitable for molecules of molecular weight around 45,000 to 50,000 amu), the latter therapeutic agent (i.e., the one with molecules of molecular weight around 45,000 to 50,000 amu) are preferably added to the reservoir prior to adding the smaller therapeutic agent (i.e., the one with molecules of molecular weight around 14,000 to 15,000 amu). Alternatively and additionally, in embodiments wherein the two different porous materials together comprise the reservoir, each carrier material may be separately loaded with a different therapeutic agent and then the carrier materials may be combined to yield the reservoir.

The therapeutic agent may be introduced into the carrier material in admixture or solution with one or more pharmaceutically acceptable excipients. The therapeutic agent may be formulated for administration in any suitable manner, such as in the form of an reservoir, suitably for subcutaneous, intramuscular, intraperitoneal or epidermal introduction or for implantation into an organ (such as the eye, liver, lung or kidney). Therapeutic agents according to the invention may be formulated for parenteral administration in the form of an injection, e.g., intraocularly, intravenously, intravascularly, subcutaneously, intramuscularly or infusion, or for injection into a synovial cavity.

In certain embodiments, the porous silicon-based carrier material is loaded with the one or more therapeutic agents at the point of service, such as in the doctor's office or hospital, prior to administration of the reservoir. For example, the porous silicon carrier material may be loaded with the therapeutic agent a short period of time prior to administration, such as 24 hours or less prior to administration, 3 hours or less prior to administration, 2 hours or less prior to administration, 1 hour or less prior to administration or 30 minutes or less prior to administration.

The reservoir may be in any suitable form prior to loading with the therapeutic agent such as in the form of a dry powder or particulate or formulated in an aqueous slurry, e.g., with a buffer solution or other pharmaceutically acceptable liquid. The therapeutic agent may be in any suitable form prior to loading into the reservoir such as in a solution, slurry, or solid such as a lyophilisate. The reservoir and/or the therapeutic agent may be formulated with other components such as excipients, preservatives, stabilizers, e.g., sugars, or therapeutic agents, e.g., antibiotic agents.

In some embodiments, the reservoir may be formulated (and packaged and/or distributed) already loaded with biomolecules, such as proteins or antibodies, while in other embodiments, the reservoir or reservoir composition is formulated (and packaged and/or distributed) essentially free of biomolecules, e.g., contains less than 5% biomolecules or less than 2% biomolecules, e.g., for combination with a biomolecule at the time of administration.

In certain embodiments, the biomolecule is a fusion protein. A fusion protein contains at least two polypeptide domains that are not ordinarily contiguous in nature. For example, the polypeptide domains may be derived from different organisms or different genes. In some embodiments, one such domain has therapeutic activity and the other domain facilitates production or improves pharmacokinetic properties. Commonly used domains in a fusion protein include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of fusion proteins by affinity chromatography. Fusion proteins may also include “epitope tags,” which are usually short peptide sequences for which a specific antibody is available, such as FLAG, influenza virus haemagglutinin (HA), and c-myc tags. In certain embodiments, the fusion polypeptides may contain one or more modifications that are capable of stabilizing the polypeptides. For example, such modifications enhance the in vitro half-life of the polypeptides, enhance circulatory half-life of the polypeptides, or reduce proteolytic degradation of the polypeptides. In certain embodiments, a linker region is positioned between two polypeptide domains. Methods for producing fusion proteins are well known. One may, for example, produce a hybrid gene such that a host cell directs expression of the fusion protein. As another example, one may produce one or more polypeptide domains separately and then covalently link the domains using a chemical cross-linker.

The therapeutic agent may be formulated (and packaged and/or distributed) as a solution with a concentration of >50 mg/mL, such as >60 mg/mL, such as >75 mg/mL. In exemplary embodiments, the therapeutic agent is bevacizumab and the bevacizumab may be formulated with a concentration of >50 mg/mL, such as >60 mg/mL, such as >75 mg/mL in, for example, a phosphate buffer solution. The therapeutic agent may be formulated (and packaged and/or distributed) with a surfactant, and/or a stabilizer, e.g., sugars, wherein the therapeutic agent has a maximum concentration of 50 mg/mL. A protein fragment, such as an antibody fragment, may be formulated (and packaged and/or distributed) as a solution with a concentration of >10 mg/mL, >15 mg/mL or >20 mg/mL.

The therapeutic agent may be formulated (and packaged and/or distributed) with stabilizers, excipients, surfactants or preservatives. In some embodiments, the stabilizers, excipients, surfactants or preservatives are sugars. In particular embodiments, the sugars are selected from trehalose, sucrose, mannitol, sorbitol, xylitol or glycerol. In other embodiments, the therapeutic agent is formulated (and packaged and/or distributed) essentially free of any one or more of stabilizers, excipients, surfactants and preservatives, e.g., contains less than 1 mg/mL or preferably less than 0.1 mg/mL of a stabilizer, excipients, surfactant or preservative. The formulation of the therapeutic agent may contain less than 1 mg/mL of surfactants such as less than 0.1 mg/mL of surfactants.

In certain embodiments, the reservoir may be sold and/or distributed preloaded in any portion of a syringe such as the barrel of a syringe or the needle of a syringe, in any suitable form, such as a dry powder or particulate, or as a slurry (e.g., in combination with a biocompatible liquid, such as an aqueous solution). The preloaded syringe may comprise other components in addition to the reservoir such as excipients, preservatives, therapeutic agents, e.g., antibiotic agents or stabilizers. The preloaded syringe may include biomolecules, such as proteins and/or antibodies, or may comprise a solution that is essentially free of biomolecules, e.g., less than 5% biomolecules or less than 2% biomolecules.

In certain embodiments, the porous silicon-based carrier material is loaded with one or more therapeutic agents within the barrel of a syringe. In particular embodiments, the reservoir is located within the barrel of a syringe as discussed above or it may be drawn up into a syringe from a separate vessel. With the reservoir in the syringe, a solution containing one or more therapeutic agents may be drawn into the syringe, thereby contacting the reservoir. Alternatively, the reservoir may be drawn up into the syringe after the therapeutic agent or a solution thereof is drawn into the barrel of the syringe. Once these components are combined, the mixture is allowed to incubate for a period of time to allow the therapeutic agent to load into the pores of the reservoir. In certain embodiments, the mixture is incubated for about 3 hours or less, about 2 hours or less, or about 1 hour or less, e.g., for about 30 minutes, about 20 minutes, about 10 minutes or about 5 minutes.

In certain embodiments, the reservoir, such as an implant, may comprise a coating surrounding the particles (e.g., the reservoir/agent/sugar complex). For example, the reservoir may be coated with a polymeric coating (e.g., by spray-drying) or an excipient such as cocoa butter. A polymeric coating may be biodegradable or non-biodegradable, permeable or non-permeable to the release of the agent. One of skill in the art will recognize that it is preferred for the polymer to be permeable, biodegradable, or both in order for the agent to be released from the particles.

In some embodiments, the reservoir comprises a coating that encloses the carrier particles, keeping the carrier localized in one area of the body. At the same time, the coating allows the agent (such as a drug) to exit the coating and reach target tissues. Thus, at least a portion of the coating may be permeable to the agent.

At least a portion of the coating may be permeable to the agent. “Permeable” denotes that the coating allows an effective amount of the agent to exit the reservoir. Certain parts of the coating may be impermeable to the agent. The term “impermeable”, as used herein, means that the coating will not allow passage of the agent at a rate required to obtain the desired local or systemic physiological or pharmacological effect, during the period when the reservoir delivers an effective amount of the agent to the subject. In some embodiments, the impermeable region has a permeability for the agent of less than 10%, 5%, 2%, 1%, 0.5%, 0.2%, 0.1%, 0.05%, 0.02%, or 0.01% of the permeability of the permeable region.

The permeability of a portion of the coating may be affected by its thickness. An impermeable coating should be thick enough not to release a significant amount of agent, relative to a permeable region of the reservoir. The thickness of an impermeable coating can be, for example, between about 0.01 and about 2 mm, preferably between about 0.01 and about 0.5 mm, most preferably between about 0.01 and about 0.2 mm. A permeable coating should be thick enough to contain the carrier particles in the tube, yet not so thick as to prevent release of an effective amount of the agent. The thickness of the permeable coating can be, for example, between about 0.01 and about 2 mm, preferably between about 0.01 and about 0.5 mm, most preferably between about 0.01 and about 0.2 mm.

The coating may also provide structure to the reservoir. The coating may be dimensionally stable and retain its shape in the absence of the particles.

In some embodiments, the coating is substantially non-biodegradable. In some embodiments, the coating does not substantially biodegrade in a biological environment prior to release of at least 90%, 95%, or 99% of the agent. In some embodiments, the coating substantially biodegrades in a biological environment after release of at least 90%, 95%, or 99% of the agent.

In some embodiments, the coating comprises a polymer. Generally speaking, suitable biocompatible polymers for use in the subject reservoirs include, but are not limited to, poly(vinyl acetate) (PVAC), poly(caprolactone) (PCL), ethylene vinyl acetate polymer (EVA), poly(ethylene glycol) (PEG), poly(vinyl alcohol) (PVA), poly(lactic acid) (PLA), poly(glycolic acid) (PGA), poly(lactic-co-glycolic acid) (PLGA), polyalkyl cyanoacrylate, polyurethane, nylons, or copolymers thereof. In polymers including lactic acid monomers, the lactic acid may be D-, L- (e.g., poly-L-lactic acid (PLLA)), or any mixture of D- and L-isomers. In some embodiments, the polymer is polyimide. In some embodiments, the polymer is PLGA that comprises lactic acid (L) and glycolic acid (G) monomers in a ratio of about 95% L and 5% G. The percentage of L may range between 80-97%. The percentage of G may range between 3-20%. In some embodiments, the polymer is heat curable, radiation curable, light (including ultraviolet) curable, evaporation curable, or curable by catalysis. In certain embodiments, the polymer is silicone, such as a silicone rubber, polydimethylsiloxane, or silicone-carbonate copolymer.

Certain polymers, like PVA, can be made more or less permeable by altering the degree of polymer cross-linking Some polymers may be permeable or impermeable depending on the relative characteristics of the polymer and the drug in the drug core. For instance, a given polymer may be permeable to a small molecule but impermeable to an antibody.

The coating may be permeable or impermeable. Exemplary polymers suitable for construction of permeable portions of the coating include PVA and PEG. Exemplary polymers suitable for construction of impermeable portions of the coating include nylons, polyurethane, EVA, polyalkyl cyanoacrylate, poly(tetrafluoroethylene) (PTFE), polycarbonate (PC), poly(methyl methacrylate) (PMMA), high grades of ethylene vinyl acetate (EVA) (e.g., 9% vinyl, content), poly(lactic-co-glycolic acid) (PLGA), and polyvinyl alcohol (PVA), especially cross-linked PVA.

In some embodiments, the coating comprises an acrylate and/or methacrylate polymer, e.g., a EUDRAGIT polymer (sold by Rohm America, Inc.). Specific EUDRAGIT polymers can be selected having various permeability and water solubility, which properties can be pH dependent or pH independent. For example, EUDRAGIT RL, EUDRAGIT NE, and EUDRAGIT RS are acrylic resins comprising copolymers of acrylic and methacrylic acid esters with a low content of quaternary ammonium groups, which are present as salts and give rise to the permeability of the lacquer films. EUDRAGIT RL is freely permeable and EUDRAGIT RS is slightly permeable, independent of pH. In contrast, the permeability of EUDRAGIT L is pH-dependent. EUDRAGIT L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable. pH sensitive coatings may be particularly useful for oral administration, for example, when the release of the agent in the intestine is preferable to the release of the agent in the stomach.

Other polymers suitable for coatings, such as ethyl cellulose and cellulose acetate, can also be used in the coating.

In certain embodiments, the coating comprises silicon (for instance, elemental silicon) or silica. A silicon or silica coating may be biodegradable.

If desired, two or more types of polymeric substances may be mixed for use as the coating. In certain embodiments, the coating may comprise one or more suitable polymers, such as a combination of two or more of the polymers discussed above.

Methods of Use

In certain embodiments, the reservoirs are used to prevent or treat a condition of a subject. The various embodiments provided herein are generally provided to deliver a therapeutically effective concentration of a therapeutic agent locally, i.e., to the site of the pain, disease, etc., in a subject. In certain embodiments, the reservoirs of the invention may be delivered to any site on the surface or within the body of a subject. For example, reservoirs of the invention may be used on the surface of the skin or eye or may be implanted under the skin, within a muscle, within an organ, adjacent to a bone, within the eye or at any other location where an equilibrium concentration of a therapeutic agent would be beneficial. The reservoir may be administered intravitreally, subcutaneously, subconjunctivally, intraperitoneally, intramuscularly, or subretinally. In certain embodiments, the reservoir of the invention is delivered to the surface of the eye or within the eye such as within the uveal tract of the eye or within the vitreous of the eye. In some embodiments, the reservoir is delivered to a synovial cavity.

In certain embodiments, the reservoirs of the invention are used to treat intraocular diseases, such as back of the eye diseases. Exemplary intraocular diseases include iritis, iridocyclitis, diffuse posterior uveitis, choroiditis, optic neuritis, chorioretinitis, and anterior segment inflammation. Other examples of intraocular diseases include glaucoma, age-related macular degeneration, such as wet age-related macular degeneration, diabetic macular edema, geographic atrophy, choroidal neovascularization, uveitis, diabetic retinopathy, retinovascular disease and other types of retinal degenerations.

In certain embodiments, the reservoirs of the invention are used to treat diseases on the surface of the eye. Exemplary diseases include viral keratitis and chronic allergic conjunctivitis. In certain embodiments, the method for treating an ocular condition comprises disposing the reservoir on the surface of the eye or within the eye such as within the vitreous or aqueous of the eye. In certain embodiments, the reservoir is injected or surgically inserted within the eye of the subject. In certain embodiments, the reservoir is injected within the eye of the subject, e.g., into the vitreous of the eye. In certain embodiments, the reservoir is injected as a composition. In certain embodiments, a reservoir composition comprises multiple reservoirs. The reservoir composition may comprise reservoirs with an average size between about 1 micron to about 500 microns. In certain embodiments, the composition comprises reservoirs with an average reservoir size between 5 microns and 300 microns, such as between about 5 microns and 100 microns.

When the beneficial substance acts on the eye, the reservoir can gradually release the beneficial substance to the eye, avoiding painful repeated administrations of a different formulation of the beneficial substance. Accordingly, the reservoir can be surgically implanted into the eye of the subject, for example the vitreous of the eye, under the retina, and onto the sclera.

The reservoir can also be inserted into numerous other locations in the body, including administration that is subcutaneous, intramuscular, intraperitoneal, intranasal, dermal, into the brain, including intracranial and intradural, into the joints, including ankles, knees, hips, shoulders, elbows, wrists, directly into tumors, and the like. For example, the reservoir can be inserted into a synovial joint.

In certain embodiments, the invention comprises a method of loading a therapeutic agent into the porous silicon-based carrier material prior to administration to a subject, such as shortly before administration to a subject. A healthcare practitioner may obtain the therapeutic agent or agents and the silicon-based carrier material, for example, together in a package as part of a kit or separately. The therapeutic agent or agents may be obtained in solution such as an aqueous or organic solution, as a lyophilisate for reconstitution, or in any other suitable form.

The practitioner may introduce the therapeutic agent or agents to the reservoir in any suitable manner, such as by incubation of the agent and the reservoir in a vial or in the barrel of a syringe, trocar, or needle. In particular embodiments, where the therapeutic agent is loaded onto the reservoir in a vial, the reservoir may be incubated with the therapeutic agent or agents or a solution thereof in the vial for a period of time, such as less than 24 hours, less than 2 hours, less than 1 hour, or even about 30 minutes or less.

In other embodiments, the reservoir is preloaded in the barrel of a syringe and the therapeutic agent or agents or a solution thereof is drawn into the syringe, forming a mixture with the reservoir. The mixture in the syringe may be allowed to incubate for a period of time such as 30 minutes or less. In certain embodiments, the particles are sterilized at one or more stages during the preparation of the reservoirs, e.g., immediately prior to administration or prior to loading the syringe. In certain embodiments, any suitable method for sterilizing the reservoirs may be used in preparation for implantation.

In certain aspects, reservoirs of the invention may be used to administer any therapeutic agent in a sustained fashion to a subject in need thereof. In certain embodiments, the methods of the invention may be used to treat a condition associated with the eye, for example, with an agent such as bevacizumab, ranibizumab, or aflibercept. The methods of the invention are not limited to ocular and intraocular use and may be used in any part of the body. For example, reservoirs of the invention may be used to administer therapeutic agents subdermally similar to the Norplant contraceptive reservoir. In other embodiments, reservoirs of the invention are used to administer biomolecules over a sustained period of time for the treatment of chronic diseases such as arthritis. For example, reservoirs of the invention may be used to deliver therapeutic agents such as etanercept, infliximab, or adalimumab to subjects in need of this therapy. The reservoirs of the invention may be located any place in the body such as within a muscle, under the skin, or in a joint, such as in a synovial cavity. The reservoir may comprise multiple small particles such as multiple particles 500 microns or less. The reservoirs may comprise larger particles such as greater than 500 microns or one or more particles greater than 1 mm in size such as greater than 10 mm.

The method of administering a therapeutic agent may comprise: a. providing a syringe preloaded with a porous silicon-based reservoir; b. contacting the reservoir with a therapeutic agent; and c. administering the reservoir to a subject. The porous silicon-based reservoir may be preloaded in any portion of the syringe such as the barrel of the syringe, an insert between the needle and the barrel, or in the needle of the syringe. The porous material may be preloaded into a portion of the syringe which may be removably coupled to other portions of a syringe, e.g., a cartridge. For example, the porous silicon material may be preloaded in an insert that can be removably attached between the barrel and the needle of a syringe wherein the remainder of the syringe parts are chosen from any commercially available syringe parts. In such embodiments, the insert may include one or more filters to prevent the particles from leaving the insert, such as a filter proximal to the point of attachment of the barrel with the porous reservoir positioned between the filter and the syringe needle. The filter may serve to contain the reservoir while being contacted with the therapeutic agent for loading the therapeutic agent into the pores of the reservoir. The filter may then be removed, reversed, bypassed or avoided so as to administer the loaded reservoir to the subject.

The porous silicon-based material may be preloaded into the needle of a syringe, the openings of which may be blocked by one or more disengageable blocks or filters that prevent the particles from exiting the needle until such time as is desired. Either before or after the reservoir has been loaded with the therapeutic agent, the block may be disengaged so as to permit administration of the loaded reservoir to the subject, e.g., through the needle. The preloaded needle may be removably coupled to any commercially available syringe barrel or may be affixed to a syringe barrel.

Step b of the method for administering a therapeutic agent described may be carried out by drawing the therapeutic agent into the syringe, such as by drawing the therapeutic agent in a mixture or solution into the syringe barrel. The therapeutic agent may be a small molecule or biomolecule. The reservoir may maintain an equilibrium concentration of the therapeutic agent in a physiological fluid over the course of up to four, six, twelve, eighteen, twenty-four, or even up to thirty months after administration. In some embodiments, the reservoir maintains an equilibrium concentration of the therapeutic agent in a physiological fluid over the course of 1 month to 6 months.

In certain embodiments, the reservoir is loaded in vivo by separately administering the reservoir and therapeutic agent to the subject. First, either the reservoir or a therapeutic agent, or a formulation containing the reservoir or a therapeutic agent, is administered to a subject. Second, the reservoir or a therapeutic agent, or a formulation containing the reservoir or a therapeutic agent, whichever was not delivered in the first step, is administered to the same site of the subject, allowing the therapeutic agent to adsorb into the pores of the reservoir. The adsorption of the therapeutic agent in the pores of the reservoir takes place over the first minutes, hours, or days after the second step, until the adsorption of the therapeutic agent in the pores of the reservoir reaches an equilibrium with the desorption of the agent from the reservoir into the surrounding environment, e.g., on the surface or within the body of a subject. Thereafter, the reservoir may release a therapeutically effective amount of the therapeutic agent over a time period that is longer than the initial re-equilibration time period, e.g., hours, days, weeks, months, or years.

In certain embodiments, multiple reservoirs are delivered to the subject such as two reservoirs, three reservoirs, four reservoirs or five reservoirs or more. The reservoirs may be substantially identical in size or composition or may have different sizes or make up, or different reservoirs or be loaded with different therapeutic agents. The multiple reservoirs may be administered to the subject simultaneously or over a period of time, and at one or more locations of the subject's body.

In certain embodiments, the therapeutic agent is released from the reservoir to maintain an equilibrium concentration in the surrounding physiological fluid over a duration of days, weeks, months, or years. In certain such embodiments the reservoir maintains an equilibrium concentration of a therapeutic agent in the surrounding physiological fluid from one day to two years, such as from two weeks to about one year, such as about one month to about one year. The reservoir may maintain an equilibrium concentration of a therapeutic agent in the surrounding physiological fluid over the course of 1 day to 12 months, such as 1 day to 6 months, such as over the course of 1 week to 3 months. In certain embodiments, the reservoir maintains an equilibrium concentration of a therapeutic agent in the surrounding physiological fluid for two years, such as with 18 months, 15 months, 12 months, 6 months, 3 months, or even 2 months. In certain embodiments, the reservoir maintains an equilibrium concentration of a therapeutic agent in the surrounding physiological fluid over the course of, for example, 1 month, 2 months, 3 months, 4 months, 5 months, 6 months, 7 months, or 8 months.

In certain embodiments, the therapeutic agent begins being released immediately after the reservoir administered. In certain embodiments, the reservoir maintains an equilibrium concentration of a therapeutic agent in the surrounding physiological fluid over the course of approximately 3 to 8 months, such as over the course of about 6 months. In certain embodiments, additional reservoirs of the invention are administered to a subject at appropriate periods to ensure a substantially continuous therapeutic effect.

The concentration of therapeutic agent in an aqueous solution may be assessed, for example, by serum and/or vitreous analyses, e.g., using ELISA.

In certain embodiments, the carrier material may completely or partially bioerode within a biological system. In certain embodiments, the carrier material may be resorbed by the biological system. In certain embodiments, the carrier material may be both bioerodible and resorbable in the biological system. In certain embodiments, the carrier material may be partially bioactive such that the material incorporates into living tissue. In some embodiments, after implantation, the carrier material does not substantially mineralize or attract mineral deposits. For instance, in some embodiments, the carrier material does not substantially calcify when placed in situ in a site where calcification is undesirable.

In certain embodiments, the carrier material may bioerode in a biological system. In certain embodiments, greater than about 80% of the carrier material will bioerode in a biological system, such as greater than about 85%, greater than about 90%, greater than about 92%, greater than about 95%, greater than about 96%, greater than about 97%, greater than about 98%, greater than about 99%, greater than 99.5%, or even greater than 99.9%. In certain embodiments, where the carrier material bioerodes, it is partially or completely resorbed.

In certain embodiments, the carrier material may substantially bioerode over the course of 1 week to 3 years. In certain embodiments, substantial bioerosion refers to erosion of greater than 95% of the carrier material. In certain embodiments, substantial bioerosion occurs over the course of about 1 month to about 2 years, such as about 3 months to 1 year. In certain embodiments, substantial bioerosion occurs within about 3 years, such as within about 2 years, within about 21 months, within about 18 months, within about 15 months, within about 1 year, within about 11 months, within about 10 months, within about 9 months, within about 8 months, within about 7 months, within about 6 months, within about 5 months, within about 4 months, within about 3 months, within about 2 months, within about 1 month, within about 3 weeks, within about 2 weeks, within about 1 week, or even within about 3 days. In certain embodiments, where the carrier material bioerodes, it is partially or completely resorbed.

In certain embodiments, the extent of bioerosion may be evaluated by any suitable technique used in the art. In exemplary embodiments, the bioerosion is evaluated through an in vitro assay to identify degradation products or in vivo histology and analysis. The biodegradability kinetics of the porous carrier material may be assessed in vitro by analyzing the concentration of the principle degradation product in the relevant physiological fluid. For porous silicon-based carrier materials in the back of the eye, for example, the degradation product may include orthosilicic acid, quantified, for example, by the molybdate blue assay, and the physiological fluid may be simulated or real vitreous humor. The biodegradability kinetics in vivo may be determined by implanting a known quantity of the porous silicon-based material into the relevant body site and monitoring its persistence over time using histology combined with, for example, standard microanalytical techniques. In some aspects, the invention relates to methods of reloading a porous drug-delivery reservoir in a subject, comprising administering to a site proximal to the reservoir an agent that has a higher affinity for the reservoir than for surrounding physiological fluid.

The agent may be selected from proteins, peptides, antibodies, carbohydrates, polymers, and polynucleotides. The agent may comprise an antibody or a fragment of an antibody. For example, the agent may be ranibizumab (the Fab fragment of an antibody), aflibercept (a fusion protein comprising the Fc fragment of an antibody), or bevacizumab (an antibody). In some embodiments, the agent is not ranibizumab or bevacizumab. In some embodiments the agent is etanercept (a fusion protein comprising the Fc fragment of an antibody), infliximab (an antibody), or adalimumab (an antibody). In some embodiments, the agent is not etanercept or adalimumab. In some embodiments, the agent is not insulin, monomeric insulin, lysozyme, or myoglobin.

In some embodiments, the agent is an antibody Fab fragment, such as ranibizumab. In some embodiments, the agent comprises an antibody Fc fragment, such as aflibercept or etanercept. In some embodiments, the agent is an antibody, such as bevacizumab, infliximab, or adalimumab.

In some embodiments, the reservoir comprises pores having an average pore size from about 15 nm to about 40 nm, and the agent has a molecular weight from about 80,000 to about 200,000 amu. The agent may have a molecular diameter from about 10 nm to about 14 nm. The reservoir may comprise pores having an average pore size from about 15 nm to about 25 nm. In some embodiments, the agent has a molecular weight from about 90,000 to about 110,000 amu or from about 140,000 to about 160,000 amu. In some embodiments, the agent has a molecular weight from about 90,000 to about 110,000 amu and the average pore size is 17 nm to 19 nm.

In some embodiments, the reservoir comprises pores having an average pore size from about 4 nm to about 16 nm, and the agent has a molecular weight from about 5,000 to about 70,000 amu. The agent may have a molecular diameter from about 2.5 nm to about 7 nm. The reservoir may comprise pores having an average pore size from about 7 nm to about 16 nm, and the agent may have a molecular weight from about 40,000 to about 60,000 amu. In some embodiments, the agent has a molecular weight from about 40,000 to about 60,000 amu and the average pore size is 7 nm to 14 nm.

In some embodiments, the reservoir comprises pores having a surface suitable for interacting with the agent through stabilizing electrostatic or hydrophobic interactions. The pores may be dimensioned to interact with the agent through stabilizing electrostatic or hydrophobic interactions with more than one surface of a pore wall at the same time. The agent may be glycosylated or pegylated and the surfaces of the pore walls have a positive electrostatic charge in the physiological fluid.

In some embodiments, the agent has an electrostatic charge in the physiological fluid and the surfaces of the pore walls have the opposite electrostatic charge in the physiological fluid. For example, the agent may have a negative electrostatic charge in the physiological fluid and the surfaces of the pore walls have a positive electrostatic charge in the physiological fluid. Alternatively, the agent may have a positive electrostatic charge in the physiological fluid and

The reservoir may comprise silicon. In certain embodiments, a portion of the reservoir is coated with a polymer, preferably to the extent that the entire surface of the reservoir is coated with the polymer.

In some embodiments, administering an agent comprises injecting the agent. In certain embodiments, the reservoir is located in the eye of the subject, and administering an agent to the subject comprises injecting the agent into said eye. Administering an agent to the subject may comprise injecting the agent into the aqueous humor of said eye. Administering an agent to the subject may comprise injecting the agent into the vitreous humor of said eye. In certain embodiments, the reservoir is located in a synovial cavity of the subject, and administering an agent to the subject comprises injecting the agent into said synovial cavity.

The subject may be selected from the group consisting of rodentia, lagomorpha, ovine, porcine, canine, feline, equine, bovine, and primate. For example, the subject may be a human.

In some aspects, the invention relates to methods for manufacturing a porous drug-delivery reservoir, comprising selecting an agent, determining a reservoir pore size that results in a desired equilibrium concentration of the agent in an aqueous solution, and loading a reservoir having pores of the determined pore size with the agent.

The reservoir may comprise silicon. The silicon may be bioerodible, and the silicon may be resorbable.

The agent may be selected from proteins, peptides, antibodies, carbohydrates, polymers, and polynucleotides. The agent may comprise an antibody or a fragment of an antibody. For example, the agent may be ranibizumab (the Fab fragment of an antibody), aflibercept (a fusion protein comprising the Fc fragment of an antibody), or bevacizumab (an antibody). In some embodiments, the agent is not ranibizumab or bevacizumab. In some embodiments the agent is etanercept (a fusion protein comprising the Fc fragment of an antibody), infliximab (an antibody), or adalimumab (an antibody). In some embodiments, the agent is not etanercept or adalimumab. In some embodiments, the agent is not insulin, monomeric insulin, lysozyme, or myoglobin.

In some embodiments, the agent is an antibody Fab fragment, such as ranibizumab. In some embodiments, the agent comprises an antibody Fc fragment, such as aflibercept or etanercept. In some embodiments, the agent is an antibody, such as bevacizumab, infliximab, or adalimumab.

In some embodiments, the reservoir comprises pores having an average pore size from about 15 nm to about 40 nm, and the agent has a molecular weight from about 80,000 to about 200,000 amu. The agent may have a molecular diameter from about 10 nm to about 14 nm. The reservoir may comprise pores having an average pore size from about 15 nm to about 25 nm. In some embodiments, the agent has a molecular weight from about 90,000 to about 110,000 amu or from about 140,000 to about 160,000 amu. In some embodiments, the agent has a molecular weight from about 90,000 to about 110,000 amu and the average pore size is 17 nm to 19 nm.

In some embodiments, the reservoir comprises pores having an average pore size from about 4 nm to about 16 nm, and the agent has a molecular weight from about 5,000 to about 70,000 amu. The agent may have a molecular diameter from about 2.5 nm to about 7 nm. The reservoir may comprise pores having an average pore size from about 7 nm to about 16 nm, and the agent may have a molecular weight from about 40,000 to about 60,000 amu. In some embodiments, the agent has a molecular weight from about 40,000 to about 60,000 amu and the average pore size is 7 nm to 14 nm.

In some embodiments, the method further comprises selecting a reservoir that comprises pores having a surface suitable for interacting with the agent through stabilizing electrostatic or hydrophobic interactions. The method may comprise selecting a reservoir comprises selecting a reservoir that comprises pores dimensioned to interact with the agent through stabilizing electrostatic or hydrophobic interactions with more than one surface of a pore wall at the same time. The method may comprise selecting an agent comprises selecting a glycosylated or pegylated agent and selecting a reservoir comprises selecting a reservoir that has a positive electrostatic charge in the physiological fluid. Additionally, the method may comprise selecting an agent comprises selecting an agent that has a net electrostatic charge in the physiological fluid and selecting a reservoir comprises selecting a reservoir that has the opposite electrostatic charge on the surfaces of the pore walls in the physiological fluid. For example, the method may comprises selecting an agent comprises selecting an agent that has a positive electrostatic charge in the physiological fluid and selecting a reservoir comprises selecting a reservoir that has a negative electrostatic charge on the surfaces of the pore walls in the physiological fluid. Alternatively, the method may comprise selecting an agent comprises selecting an agent that has a negative electrostatic charge in the physiological fluid and selecting a reservoir comprises selecting a reservoir that has a positive electrostatic charge on the surfaces of the pore walls in the physiological fluid.

The method may comprise coating the reservoir with a polymer after loading the reservoir.

In some embodiments, the solution is the vitreous humor of an eye. In other embodiments, the solution is the aqueous humor of an eye. Still, in other embodiments, the solution is the synovial fluid of a synovial joint.

The method may further comprise loading the reservoir with an amorphous sugar. The amorphous sugar may be selected from trehalose, trehalose dihydrate, sucrose, mannitol, sorbitol, xylitol or glycerol, or a combination thereof.

In some aspects, the invention relates to methods of delivering a therapeutically effective concentration of an agent to a site in a subject, comprising administering to the site a porous drug-delivery reservoir loaded with the agent, wherein the reservoir has pores configured to maintain a therapeutically effective equilibrium concentration of the agent at the site.

The reservoir may comprise silicon. The silicon may be bioerodible, and the silicon may be resorbable.

The agent may be selected from proteins, peptides, antibodies, carbohydrates, polymers, and polynucleotides. The agent may comprise an antibody or a fragment of an antibody. For example, the agent may be ranibizumab (the Fab fragment of an antibody), aflibercept (a fusion protein comprising the Fc fragment of an antibody), or bevacizumab (an antibody). In some embodiments, the agent is not ranibizumab or bevacizumab. In some embodiments the agent is etanercept (a fusion protein comprising the Fc fragment of an antibody), infliximab (an antibody), or adalimumab (an antibody). In some embodiments, the agent is not etanercept or adalimumab. In some embodiments, the agent is not insulin, monomeric insulin, lysozyme, or myoglobin.

In some embodiments, the agent is an antibody Fab fragment, such as ranibizumab. In some embodiments, the agent comprises an antibody Fc fragment, such as aflibercept or etanercept. In some embodiments, the agent is an antibody, such as bevacizumab, infliximab, or adalimumab.

In some embodiments, the reservoir comprises pores having an average pore size from about 15 nm to about 40 nm, and the agent has a molecular weight from about 80,000 to about 200,000 amu. The agent may have a molecular diameter from about 10 nm to about 14 nm. The reservoir may comprise pores having an average pore size from about 15 nm to about 25 nm. In some embodiments, the agent has a molecular weight from about 90,000 to about 110,000 amu or from about 140,000 to about 160,000 amu. In some embodiments, the agent has a molecular weight from about 90,000 to about 110,000 amu and the average pore size is 17 nm to 19 nm.

In some embodiments, the reservoir comprises pores having an average pore size from about 4 nm to about 16 nm, and the agent has a molecular weight from about 5,000 to about 70,000 amu. The agent may have a molecular diameter from about 2.5 nm to about 7 nm. The reservoir may comprise pores having an average pore size from about 7 nm to about 16 nm, and the agent may have a molecular weight from about 40,000 to about 60,000 amu. In some embodiments, the agent has a molecular weight from about 40,000 to about 60,000 amu and the average pore size is 7 nm to 14 nm.

In some embodiments, the reservoir comprises pores having a surface suitable for interacting with the agent through stabilizing electrostatic or hydrophobic interactions. The pores may be dimensioned to interact with the agent through stabilizing electrostatic or hydrophobic interactions with more than one surface of a pore wall at the same time. The agent may be glycosylated or pegylated and the surfaces of the pore walls have a positive electrostatic charge in the physiological fluid.

In some embodiments, the agent has an electrostatic charge in the physiological fluid and the surfaces of the pore walls have the opposite electrostatic charge in the physiological fluid. For example, the agent may have a negative electrostatic charge in the physiological fluid and the surfaces of the pore walls have a positive electrostatic charge in the physiological fluid. Alternatively, the agent may have a positive electrostatic charge in the physiological fluid and

In certain embodiments, a portion the reservoir is coated with a polymer, preferably to the extent that the entire surface of the reservoir is coated with the polymer.

In some embodiments, administering an agent comprises injecting the reservoir. In certain embodiments, and administering a reservoir to the subject comprises injecting the reservoir into said eye. Administering a reservoir to the subject may comprise injecting the reservoir into the aqueous humor of said eye. Administering a reservoir to the subject may comprise injecting the reservoir into the vitreous humor of said eye. In certain embodiments, administering a reservoir to the subject comprises injecting the reservoir into said synovial cavity.

The subject may be selected from the group consisting of rodentia, lagomorpha, ovine, porcine, canine, feline, equine, bovine, and primate. For example, the subject may be a human.

In some embodiments, the reservoir is loaded with an amorphous sugar. The amorphous sugar may be selected from trehalose, trehalose dihydrate, sucrose, mannitol, sorbitol, xylitol or glycerol, or a combination thereof.

The subject may be selected from the group consisting of rodentia, lagomorpha, ovine, porcine, canine, feline, equine, bovine, and primate. For example, the subject may be a human.

In some embodiments the reservoir is configured to maintain a therapeutically effective equilibrium concentration of the agent at the site for at least 1, 2, 3, 4, 5, 6, 7, or 8 weeks. The reservoir may be configured to maintain a therapeutically effective equilibrium concentration of the agent at the site for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months. For example, the reservoir may be configured to maintain a therapeutically effective equilibrium concentration of the agent at the site for at least 1, 2, 3, or 4 years. In some embodiments, the reservoir is configured to maintain a therapeutically effective equilibrium concentration of the agent at the site for a period of time between 2 weeks and 4 years, such as between 2 months and 3 years, or between 6 and 30 months.

The invention now being generally described, it will be more readily understood by reference to the following examples which are included merely for purposes of illustration of certain aspects and embodiments of the present invention, and are not intended to limit the invention.

### EXAMPLES

### Materials

### Specifications of Commercial Porous Silica

| | | Nominal | | |
|---|---|---|---|---|
| | | Pore Size | Surface Area | Pore Volume |
| Supplier | Trade Name | (Å) | (m²/g) | (mL/g) |
| Grace Davison | Davisil | 60 | 550 | 0.9 |
| Discovery | | 150 | 330 | 1.2 |
| Sciences | | 250 | 285 | 1.8 |
| | | 500 | 80 | 1.1 |
| | | 1000 | 40 | 1.1 |
| SiliCycle | SiliaSphere PC | 300 | 100 | 1.1 |

### Example 1

To establish the relationship between protein size and the required pore size to facilitate drug loading, the amount of surface area occupied by a protein when adsorbed at monolayer coverage was correlated to the cumulative surface area against pore size data resulting from the Barrett-Joyner-Halenda (BJH) analysis from nitrogen sorption data. The point at which the protein adsorption surface area data became equivalent to the cumulative total surface area from nitrogen sorption analysis defined the minimum accessible pore size to facilitate adsorption loading. The data in Table 1 presents the minimum pore radii accessible for a range of protein sizes. Subtracting the protein hydrodynamic radius from the minimum pore radius generates the protein-pore differential which is the minimum amount of additional pore dimension required to allow protein access. For the range of proteins investigated the average protein-pore differential was 3.9 nm.

TABLE 1
Correlation between protein size and pore accessibility

| | Hydrodynamic | Minimum Pore | Protein-Pore |
|---|---|---|---|
| | Radius | Radius | Differential |
| Protein | (nm) | (nm) | (nm) |
| Insulin (monomer) | 1.3 | 4.8 | 3.5 |
| Lysozyme | 1.9 | 5.7 | 3.8 |
| Myoglobin | 2.2 | 5.5 | 3.3 |
| Bevacizumab | 7.0 | 12.0 | 5.0 |
| | | Average | 3.9 |

### Example 2

The kinetics of bevacizumab adsorption into Davisil 250 Å was established by incubating 5 mg of the adsorbent with 25 μL of 25 mg/mL bevacizumab in phosphate buffer pH 6.2 (Table 2). After a defined equilibration time, 1.975 mL of phosphate buffer was added to the suspension and mixed by inversion for no longer than 30 seconds and the particles removed by filtration through a 0.2 μm filter. The amount of protein in the filtrate was the quantified using the BCA assay (Thermo Scientific, USA). The amount of protein adsorbed was calculated by subtracting the amount in the filtrate from the starting concentration. Table 2 presents the kinetics of adsorption for a range of particle sizes. The smallest particle size of 8.4 μm (D₅₀) resulted in 95.6% adsorption within 30 minutes compared to 73.7% and 19.8% for of 15.8 μm (D₅₀) and of 54.5 μm (D₅₀) respectively.

TABLE 2
Kinetics of bevacizumab adsorption for adsorbents
of increasing particle size

| Equilibration | Particle Size (D₅₀; μm) | | |
|---|---|---|---|
| Time | 8.4 | 15.8 | 54.5 |
| (Hours) | % Bevacizumab Adsorbed | | |
| 0.01 | 44.6 | 31.5 | 12.1 |
| 0.5 | 95.6 | 73.7 | 19.8 |
| 1 | 94.9 | 80.0 | 23.6 |
| 2 | 98.1 | 82.5 | 30.0 |
| 4 | 98.8 | 93.2 | 58.2 |
| 6.5 | 99.0 | 96.8 | 67.2 |
| 24 | 99.5 | 99.1 | 87.2 |

### Example 3

Adsorption isotherms were generated by equilibrating 1 mL of chicken egg white lysozyme (Sigma) at concentrations ranging from 270 μM to 1 μM in 50 mM phosphate buffer pH 6.2 with 5 mg of adsorbent. After 16 hours the amount of lysozyme remaining in the equilibration solution was quantified by UV spectroscopy at 280 nm. The amount of lysozyme adsorbed onto the adsorbent was then plotted against the equilibration concentration. The monolayer amount of lysozyme adsorbed and the Langmuir adsorption coefficient (K) were estimated by using standard linear transformation methods.

To measure the extent and rate of lysozyme release, the adsorbent matrices were equilibrated at room temperature for 16 hours with chicken egg white lysozyme (Sigma) in 50 mM phosphate buffer pH 6.2 (Table 3). The amount of lysozyme released into 2 mL of phosphate buffered saline (pH 7.4) saturated with SiO₂ was measured over time. At each time point the samples were centrifuged at 16,300 g and 1 mL of the supernatant removed and replaced with fresh media. The amount of lysozyme released was then quantified by high pressure liquid chromatography. The rate of lysozyme release correlated with the pore size of the adsorbent and also the strength of the interaction between lysozyme and the adsorbent as determined by the Langmuir adsorption coefficient (K).

TABLE 3
Relationship between adsorbent pore size, Langmuir
adsorption coefficient and lysozyme release rate.

| | Langmuir Coefficient | Lysozyme Release Rate |
|---|---|---|
| Adsorbent | (μM⁻¹) | (%/day) |
| Davisil 60 Å | 0.238 | 0.53 |
| Davisil 150 Å | 0.107 | 2.45 |
| Davisil 250 Å | 0.069 | 3.67 |
| Davisil 500 Å | 0.030 | 13.38 |

### Example 4

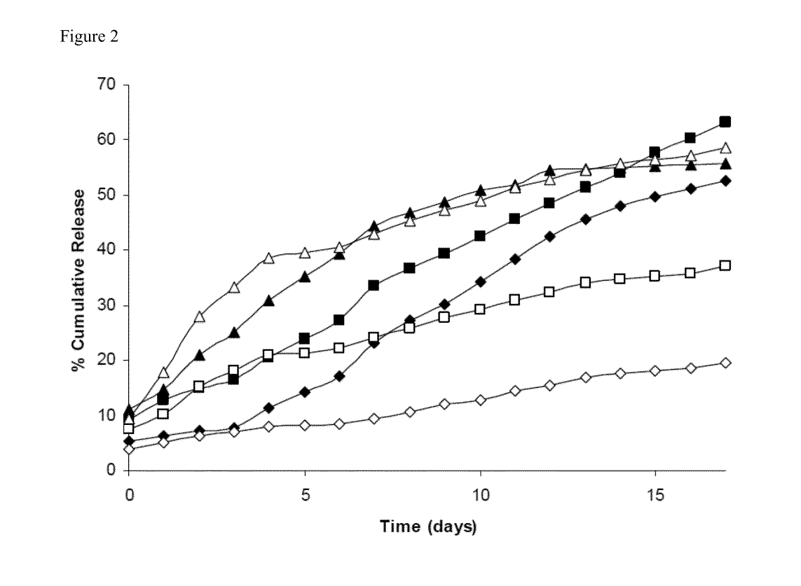

Lysozyme (Chicken egg white, Sigma) was adsorption loaded on to silica adsorbents of increasing pore size by equilibrating 50 μL, of a 25 mg/mL solution with 10 mg of adsorbent. After 16 hours, 3.95 mL of phosphate buffered saline (PBS; pH 7.4)) or phosphate buffered saline saturated with SiO₂ was added and the suspension incubated at 37° C. At each time point the particles were sedimented via centrifugation at 16,300 g and 2 mL of the supernatant was removed and replaced with 2 mL of fresh media. The amount of lysozyme in the dissolution media was then quantified by RP-HPLC. The kinetics of lysozyme release were determined by regression analysis of the cumulative release against square root time. The results are presented in FIG. 2 and Table 4.

Adsorption isotherms were generated by equilibrating 1 mL of chicken egg white lysozyme (Sigma) at concentrations ranging from 270 μM to 1 μM in 50 mM phosphate buffer pH 6.2 with 5 mg of adsorbent. After 16 hours the amount of lysozyme remaining in the equilibration solution was quantified by UV spectroscopy at 280 nm. The amount of lysozyme adsorbed onto the adsorbent was then plotted against the equilibration concentration. The monolayer amount of lysozyme adsorbed and the Langmuir adsorption coefficient (K) were estimated by using standard linear transformation methods.

Experiments were performed in both phosphate buffered saline and SiO₂ saturated phosphate buffered saline to demonstrate that lysozyme release proceeds by two mechanisms. Saturating the phosphate buffered saline with SiO₂ prevents the dissolution of the porous silica matrix. Therefore, any release of lysozyme occurred via a desorption process. In phosphate buffered saline lysozyme release resulted from a combination of matrix associated dissolution and desorption. The results in Table 4 demonstrate that there was a concomitant increase in the lysozyme desorptive release component with increasing matrix pore size. It is hypothesised that the rate of lysozyme desorption is inversely proportional to the strength of adsorption between lysozyme and the porous matrix as determined by the Langmuir coefficient.

TABLE 4
Release rate of lysozyme from silica adsorbents in phosphate buffered
saline and SiO₂ saturated phosphate buffered saline

| | Lysozyme Release | | % | Langmuir |
|---|---|---|---|---|
| | (%/day^{1/2}) | | Desorptive^{a} | Coefficient |
| Adsorbent | PBS | SiO₂ saturated PBS | Component | (μM⁻¹) |
| Davisil 60 Å | 20.9 | 6.6 | 31.6 | 0.238 |
| Davisil 150 Å | 19.8 | 8.4 | 42.4 | 0.107 |
| Davisil 250 Å | 15.8 | 12.5 | 79.1 | 0.069 |

^{a}% Desorptive component, lysozyme release in SiO₂ saturated PBS/lysozyme release in PBS × 100

### Example 5

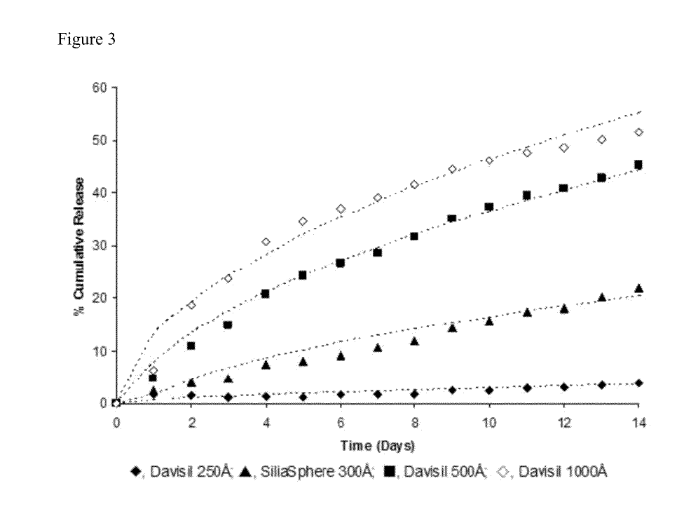

Bevacizumab was adsorbed onto silica adsorbents of increasing pore size by equilibrating 25 μL of a 25 mg/mL solution with 5 mg of adsorbent. After 16 hours 1.975 mL of phosphate buffered saline (pH 7.4) was added and the suspension incubated at 37° C. At each time point the particles were removed via centrifugation at 16,300 g and 1 mL of the supernatant was removed and replaced with 1 mL of fresh media (FIG. 3, Table 5). The amount of bevacizumab in the dissolution media was then quantified by the Micro BCA assay (Thermo Scientific, USA). The kinetics of bevacizumab release were determined by regression analysis of the cumulative release against square root time. The results demonstrate that the rate of bevacizumab release increased with increasing pore size of the adsorbent.

TABLE 5
Release rate of bevacizumab from silica adsorbents
in phosphate buffered saline

| | Bevacizumab Release |
|---|---|
| Adsorbent | (%/day^{1/2}) |
| Davisil 250 Å | 1.03 |
| SiliaSphere 300 Å | 6.78 |
| Davisil 500 Å | 13.23 |
| Davisil 1000 Å | 15.42 |

### Example 6

The kinetics of protein adsorption into porous silica of various pore size were established by incubating 5 mg of the adsorbent with 25 μL of 25 mg/mL protein solution in phosphate buffer pH 6.2. After a defined amount of equilibration time, 1.975 mL of phosphate buffer was added to the suspension and mixed by inversion for no longer than 30 seconds and the particles removed by filtration through a 0.2 μm filter. The amount of protein in the filtrate was the quantified using either the BCA assay (Thermo Scientific, USA) in the case of bevacizumab, and RP-HPLC for lysozyme. The amount of protein adsorbed was calculated by subtracting the amount in the filtrate from the starting concentration. Tables 5a, 5b, 6a and 6b present the kinetics of adsorption for a range of porous silica pore sizes and particle sizes.

For both lysozyme and bevacizumab it was evident that an increase in matrix pore size resulted in a faster rate of protein adsorption. The results in Tables 6a and 6b also demonstrate that a decrease in particle size resulted in an increased rate of protein adsorption.

TABLE 5a
The effect of pore size on lysozyme adsorption.

| | Lysozyme adsorption | | |
|---|---|---|---|
| | (μg/mg silica) | | |
| Time | Porous silica (Å) | | |
| (hours) | 60 | 250 | 1000 |
| 0.01 | 68.3 | 100.3 | 50.8 |
| 0.5 | 84.9 | 100.6 | 48.9 |
| 1 | 93.3 | 103.0 | 49.2 |
| 2 | 98.2 | 104.2 | 46.6 |
| 4 | 107.6 | 101.5 | 47.7 |
| 6 | 109.6 | 102.6 | 51.2 |
| 24 | 116.5 | 103.3 | 51.9 |

TABLE 5b
The effect of pore size on normalised lysozyme loading.

| | Lysozyme % normalised | | |
|---|---|---|---|
| | loading (%)^{a} | | |
| Time | Porous silica (Å) | | |
| (hours) | 60 | 250 | 1000 |
| 0.01 | 59 | 97 | 98 |
| 0.5 | 73 | 97 | 94 |
| 1 | 80 | 100 | 95 |
| 2 | 84 | 101 | 90 |
| 4 | 92 | 98. | 92 |
| 6 | 94 | 99 | 99 |
| 24 | 100. | 100 | 100 |

^{a}Lysozyme normalised loading (%) is the amount of lysozyme adsorbed (μg/mg)/the amount of lysozyme adsorbed at 24 hours × 100

TABLE 6a
The effect of pore size and particle size on bevacizumab
adsorption.

| Bevacizumab adsorption (μg/mg silica) | | |
|---|---|---|
| Porous silica (Å) | | |
| 500 | 300 | 250 |

| | |
|---|---|
| Time | Particle size (D₅₀; μm) |

| (hours) | 77 | 45 | 54 | 19 | 16 | 8 |
|---|---|---|---|---|---|---|
| 0.01 | 34.9 | 8.6 | 15.2 | 53.7 | 39.4 | 55.8 |
| 0.5 | 64.4 | 59.5 | 24.8 | 76.3 | 92.1 | 119.5 |
| 1 | 71.8 | 62.9 | 29.5 | 75.3 | 100.0 | 118.6 |
| 2 | 86.7 | 91.0 | 37.5 | 98.0 | 103.1 | 122.6 |
| 4 | 91.5 | 109.7 | 72.8 | 103.8 | 116.5 | 123.5 |
| 6 | 97.0 | 107.9 | 83.9 | 107.9 | 121.0 | 123.8 |
| 24 | 108.4 | 122.6 | 109.0 | 118.7 | 123.9 | 124.4 |

TABLE 6b
The effect of pore size and particle size on normalised
bevacizumab loading.

| Bevacizumab % normalised loading (%)^{a} | | |
|---|---|---|
| Porous silica (Å) | | |
| 500 | 300 | 250 |

| | |
|---|---|
| Time | Particle size (D₅₀; μm) |

| (hours) | 77 | 45 | 54 | 19 | 16 | 8 |
|---|---|---|---|---|---|---|
| 0.01 | 32 | 7 | 14 | 45 | 32 | 45 |
| 0.5 | 59 | 49 | 23 | 64 | 74 | 96 |
| 1 | 66 | 51 | 27 | 63 | 81 | 95 |
| 2 | 80 | 74 | 34 | 83 | 83 | 99 |
| 4 | 84 | 90 | 67 | 87 | 94 | 99 |
| 6 | 89 | 88 | 77 | 91 | 98 | 99 |
| 24 | 100 | 100 | 100 | 100 | 100 | 100 |

^{a}Bevacizumab normalised loading (%) is the amount of bevacizumab adsorbed (μg/mg)/the amount of bevacizumab adsorbed at 24 hours × 100

### Example 7

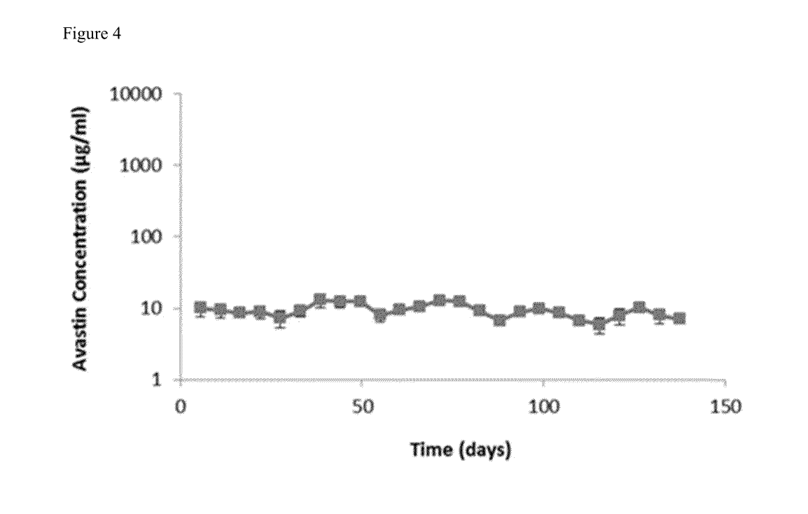

Silicon was prepared with a surface area of 205 m²/g, pore volume of 0.899 ml/g, pore diameter of 17.5 nm, and loading of 8-10% w/w. 50 μl of bevacizumab injection solution (25 mg/ml) was added to 10 mg silicon and incubated for 30 min. 4 ml of PBS was then added onto bevacizumab/BioSilicon matrix, and the suspension was incubated in a 37° C. water bath. 25 samples were taken periodically by centrifuging the suspension, removing 2 ml of clear supernatant for analysis, and replacing the 2 ml aliquot with 2 ml of fresh buffer. The bevacizumab concentration was maintained at an equilibrium concentration, 9.4±2.0 μg/ml, as shown in FIG. 4.

### Example 8

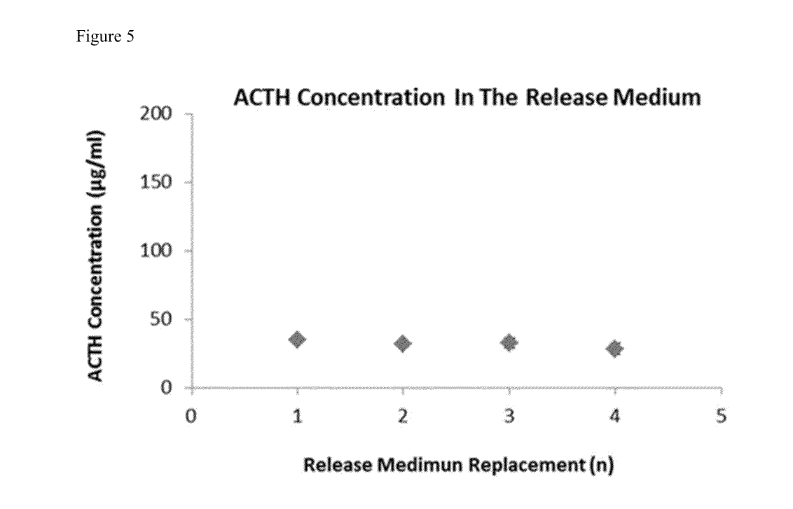

A reservoir was loaded with adrenocorticotropic hormone (ACTH) and placed in 4 ml of aqueous solution. 2 ml of the 4 ml aqueous solution was removed at periodic time points and replaced with fresh solution. The silicon reservoir maintains an ACTH concentration of 30±4.5 μg/ml (FIG. 5).

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. A method of reloading a porous drug-delivery reservoir in a subject, comprising administering to a site proximal to the reservoir an agent that has a higher affinity for the reservoir than for surrounding physiological fluid.

2. The method of claim 1, wherein the agent is selected from proteins, peptides, antibodies, carbohydrates, polymers, and polynucleotides.

3. The method of claim 5, wherein the agent comprises an antibody Fc fragment and the agent is etanercept.

4. (canceled)

5. The method of claim 2, wherein the agent comprises an antibody or a fragment of an antibody.

6. (canceled)

7. The method of claim 5, wherein the agent is an antibody and the antibody is bevacizumab, infliximab, or adalimumab.

8. The method of claim 1, wherein the reservoir comprises pores having an average pore size from about 15 nm to about 40 nm, and the agent has a molecular weight from about 80,000 to about 200,000 amu.

9. The method of claim 8, wherein the agent has a molecular diameter from about 10 nm to about 14 nm and the reservoir comprises pores having an average pore size from about 15 nm to about 25 nm.

10. (canceled)

11. The method of claim 8, wherein the agent has a molecular weight from about 90,000 to about 110,000 amu and the reservoir comprises pores having an average pore size from about 15 nm to about 25 nm.

12. The method of claim 8, wherein the agent has a molecular weight from about 140,000 to about 160,000 amu and the reservoir comprises pores having an average pore size from about 15 nm to about 25 nm.

13-14. (canceled)

15. The method of claim 5, wherein the agent is an antibody Fab fragment and the antibody Fab fragment is ranibizumab.

16. The method of claim 1, wherein the reservoir comprises pores having an average pore size from about 4 nm to about 16 nm, and the agent has a molecular weight from about 5,000 to about 70,000 amu.

17. The method of claim 1, wherein the reservoir comprises pores having an average pore size from about 4 nm to about 16 nm and the agent has a molecular diameter from about 2.5 nm to about 7 nm.

18. The method of claim 16, wherein the reservoir comprises pores having an average pore size from about 7 nm to about 16 nm, and the agent has a molecular weight from about 40,000 to about 60,000 amu.

19. The method of claim 1, wherein the reservoir comprises pores having a surface suitable for interacting with the agent through stabilizing electrostatic or hydrophobic interactions.

20-21. (canceled)

22. The method of claim 19, wherein the agent has an electrostatic charge in the physiological fluid and the surfaces of the pore walls have the opposite electrostatic charge in the physiological fluid.

23-24. (canceled)

25. The method of claim 1, wherein the reservoir comprises silicon.

26. The method of claim 1, wherein a portion the reservoir is coated with a polymer.

27. The method of claim 1, wherein administering an agent comprises injecting the agent.

28. The method of claim 27, wherein the reservoir is located in an eye of the subject, and administering an agent to the subject comprises injecting the agent into the eye.

29. The method of claim 28, wherein administering an agent to the subject comprises injecting the agent into the aqueous humor of the eye or the vitreous humour of the eye.

30. (canceled)

31. The method of claim 1, wherein the reservoir is located in a synovial cavity of the subject, and administering an agent to the subject comprises injecting the agent into the synovial cavity.

32. The method of claim 1, wherein the subject is selected from rodentia, lagomorpha, ovines, porcines, canines, felines, equines, bovines, and primates.

33. The method of claim 32, wherein the subject is a human.

34. A method for manufacturing a porous drug-delivery reservoir, comprising selecting an agent, determining a reservoir pore size that results in a desired equilibrium concentration of the agent in an aqueous solution, and loading a reservoir having pores of the determined pore size with the agent.

35-66. (canceled)

67. A method of delivering a therapeutically effective concentration of an agent to a site in a subject, comprising administering to the site a porous drug-delivery reservoir loaded with the agent, wherein the reservoir has pores configured to maintain a therapeutically effective equilibrium concentration of the agent at the site.

68-109. (canceled)
